(19) United States
(12) Patent Application Publication
(10) Pub. No.: US 2023/0270763 A1
(43) Pub. Date: Aug. 31, 2023

(54) METHOD OF TREATING CORONOVIRUS INFECTIONS WITH CANNABINOIDS AND DERIVATIVES

(71) Applicant: PASCAL BIOSCIENCES INC., Vancouver (CA)

(72) Inventor: Thomas L. Deckwerth, Seattle, WA (US)

(21) Appl. No.: 18/014,169

(22) PCT Filed: Jul. 2, 2021
(86) PCT No.: PCT/US2021/040254

§ 371 Date: Jan. 2, 2023

Related U.S. Application Data
(60) Provisional application No. 63/047,311, filed on Jul. 2, 2020.

(51) Int. Cl. A61K 31/00; A61K 45/06; A61P 31/14

(52) U.S. Cl. CPC A61K 31/658; A61K 45/06; A61P 31/14

(57) ABSTRACT

This disclosure relates to a method of treating corona virus infections. More specifically disclosed are cannabinoids and derivatives effective for the treatment of RNA viral infections.

### FIELD OF THE INVENTION

This disclosure relates to a method of treating coronavirus infections. More specifically disclosed are cannabinoids and derivatives effective for the treatment of viral infections.

### BACKGROUND OF THE INVENTION

Viral infections cause various impacts from the discomfort of the rhinovirus to the significant loss of life associated with HIV, hepatitis and yellow fever. Recently, coronavirus infections have caused millions of deaths worldwide.

The novel coronavirus designated SARS-CoV-2 spread from China in late 2019, inducing a global pandemic of severe acute respiratory disease, referred to as COVID-19 (Zhou M et al. Coronavirus disease 2019 (COVID-19): a clinical update Front Med. 2020 Apr. 2: 1-10). The most severe cases of COVID-19 are characterized by intense and dysregulated inflammation, pneumonia, lung damage, and severe respiratory distress, requiring high-pressure oxygen therapy and eventually prolonged ventilation. The prognosis for elderly patients with severe or critical disease is poor, with a reported mortality rate close to 20% (Wang L et al. Coronavirus Disease 2019 in elderly patients: characteristics and prognostic factors based on 4-week follow-up. J Infect. 2020 Mar. 30 doi: 10.1016/j.jinf.2020.03.019 [Epub ahead of print]). Surviving patients can also be expected to have lasting damage as a result of their severe inflammatory response. Damage induced in lung tissue and other organs by SARS-CoV-2 is driven primarily by a cytokine response syndrome or “cytokine storm” involving intense release of high levels of pro-inflammatory cytokines such as interleukin-6 (IL-6) from cells of the host (McGonagle D et al. Interleukin-6 use in COVID-19 pneumonia related macrophage activation syndrome. Autoimmun Rev. 2020 Apr. 3:102537. doi: 10.1016/j.autrev.2020.102537 [preprint]). The excessive pro-inflammatory cytokine release induced by SARS-CoV-2 can lead to multi-organ failure, a significant cause of death for infected patients with severe disease.

In 2003, SARS-CoV was identified as a new emerging infectious pathogen responsible for severe acute respiratory syndrome in humans. During winter 2002/2003, more than 8000 people were infected by this highly infectious agent and 10% of them died. The SARS-CoV mainly targets the epithelial cells, the respiratory tract being the primary site of infection. One of the major pathological features of SARS-CoV infection is diffuse alveolar damage (DAD) of the human lung, more prominent in the terminal stage, with sometimes, abrupt deterioration of the lung epithelium. The SARS pathogen triggered atypical pneumonia characterized by high fever, severe dyspnea and the development of acute severe king failure.

There has been recent publications in 2021 related to this invention. In March 2021, Nguyen et al. reported that cannabidiol inhibits SARS-CoV-2 replication and promotes the host innate immune response [bioRx 2021. Https://doi.org/10.1101/2021.03.10.432967]. Additionally, Raj et al. described potencies of some cannabinoids against SARS-CoV-2 [Int. J Biol. Macromolecules 168 2021 (474-85)].

The inventors have identified therapeutically useful cannabinoids for the preparation of a medicament for the treatment of a subject with coronavirus infection.

### BRIEF SUMMARY OF THE INVENTION

One aspect of the invention is a method of treating a subject infected or suspected of being infected with an RNA virus with a cannabinoid compound or derivative thereof. One aspect of the present invention is a method of relieving a symptom of an RNA virus infection in a subject with a cannabinoid compound or derivative thereof. An aspect of the current invention involves treating subjects with coronavirus infections with a cannabinoid or cannabinoid derivative. Also disclosed is a treatment of a subject exposed to COVID-19 with a cannabinoid or cannabinoid derivative. Also disclosed is a method of treating an RNA virus-related disease in a subject, comprising administering to the subject a cannabinoid compound, or derivative thereof.

Various embodiments of this invention relate to use of a cannabinoid or cannabinoid derivative in preparation of a medicament for treating RNA virus-related disease.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. shows the percent inhibition of virus-induced cell death by CBD in SARS-CoV-2-infected cells.

FIG. 2. shows the cell viability of CBD-treated non-infected cells.

### DESCRIPTION OF THE PREFERRED EMBODIMENTS

The embodiments listed below may be presented in numbered form for convenience and in ease and clarity of reference in referring back to multiple embodiments.

One aspect of the invention is a method of treating a subject infected or suspected of being infected with an RNA virus with a cannabinoid compound or derivative thereof.

One aspect of the present invention is a method of relieving a symptom of an RNA virus-infection in a subject with a cannabinoid compound or derivative thereof.

An aspect of the current invention involves treating subjects with coronavirus infections with a cannabinoid or cannabinoid derivative.

Also disclosed is a treatment of a subject exposed to COVID-19 with a cannabinoid or cannabinoid derivative.

Also disclosed is a method of treating an RNA virus-related disease in a subject, comprising administering to the subject a cannabinoid compound, or derivative thereof.

The disclosure provides compounds effective in impacting cell viability and cytoprotection in the presence of RNA virus-infection. For example, in some aspects, the compounds are capable of preventing cell damage, improving cell survival, and/or enhancing resistance to viral stress. Compounds that are cytoprotective have potential utility to extend the viability of cells in culture.

SARS-CoV-2 is a member of the β genus of the Coronavirus family, along with the other human pathogens HCoV-HKU1, HCoV-OC43, SARS-CoV, and MERS-CoV, the bovine coronavirus BCoV and mouse hepatitis virus MHV. Members of the α genus include human pathogens HCoV-NL63 and HCoV-229E as well as the porcine viruses TGEV and PRCV. Avian infectious bronchitis virus (IBV) is a member of the γ genus. Coronavirus virions possess a large single-stranded and positive-sense RNA genome, termed (+)ssRNA, of typically between 27 and 32 kb, packaged into a helical capsid and envelope, The envelope is decorated with protruding spikes which interact with host cell receptors to allow cell fusion and infection. The receptor differs between different coronaviruses, with HCoV-NL63, SARS-CoV and SARS-CoV-2 utilizing angiotensin converting enzyme 2 (ACE2), while other coronaviruses use other cell surface molecules for docking. Coronaviruses exhibit a wide range of pathologies depending on strain, host species and physiological status of the host. Coronaviruses OC43, 229E, NL63, and HKU1 usually just cause mild to moderate illness of the upper-respiratory tract, like the common cold. The severe acute respiratory syndrome (SARS) epidemic in 2003 was caused by SARS-CoV, a highly contagious virus causing respiratory disease with the lungs as the major target. In severe cases, SARS-CoV infection was also characterized by an intense, dysregulated local inflammatory response leading to devastating lung pathology. SARS-CoV-2 infection impacts the body in two related processes. The first is the harm the virus wreaks on blood vessels, leading to blood clots of various sizes that may cause strokes and pulmonary emboli as clots break loose and travel to the brain and lungs. This may be because the virus directly targets the endothelial cells that line blood vessels. The second is an exaggerated and dysregulated response from the body's own immune system, a storm of killer “cytokines” that attack the body's own cells along with the virus as it seeks to defend the body from an invader. Other coronaviruses may affect other organs, for example different strains of MH may cause liver pathology or encephalitis in mice. The deadliest coronavirus disease appears to be MERS with a lethargy of over 30% of all laboratory-confirmed cases.

According to the preferred embodiments, the cannabinoid is a bio-acceptable substance such as cannabidiol, a phytocannabinoid, a cannabinoid mimetic, or a cannabinoid derivative thereof, or pharmaceutical compositions made therefrom. According to specific preferred embodiments, the substance is a novel compound with structural relationship to a cannabinoid. According to additional specific preferred embodiments, the substance is a novel compound with additional modifications beyond structural relationship to a cannabinoid. Such modifications may enable improved biochemical or pharmaceutical properties.

While it is not intended that the present invention should be limited by any particular theory of action or biochemical mechanisms by which it is believed to operate, the following is offered and postulated for a better understanding of the invention and its practice.

Cannabinoids are prenylated polyketides derived from fatty acid and isoprenoid precursors. These secondary metabolites are synthesized primarily in glandular trichomes of female flowers and, to a lesser extent, in leaves of various Cannabis strains and some other plants. Their biosynthesis involves the enzymatically catalyzed joining of alkylresorcinol and monoterpene moieties by dedicated transferases and synthases as well as non-enzymatic reactions resulting in several structurally distinguishable classes. These phytocannabinoids may act on cannabinoid receptors and on additional cellular targets such as other G protein-coupled receptors, ion channels, transporters, and enzymes in the brain and other tissues (Soderstrom et al., Frontiers Pharmacol. 8:1-28, 2017) to effect a variety of physiological responses beyond the commonly known psychoactive properties of some of the tetrahydrocannabinoids. The endocannabinoids, such as anandamide, are produced naturally in the body and act as natural ligands for cannabinoid receptors (Devane et al., Science 258:1946-1949, 1992). Synthetic cannabinoids are manufactured artificially, have activity on cannabinoid receptors, and many have structural similarity to natural cannabinoids. Perhaps the most notable cannabinoid is tetrahydrocannabinol (THC), the primary psychoactive compound in Cannabis. More than 100 different cannabinoids have been isolated from Cannabis.

Mammalian viruses are currently classified in seven groups based on the nature of their genome: (−)ssRNA, (+)ssRNA, dsRNA, ssRNA-RT, ssDNA, dsDNA and dsDNA-RT viruses. Examples of (−)ssRNA viruses include Junin virus, Lassa virus, Lymphocytic choriomeningitis virus, Ebola virus, Marburg virus, Ravn virus, Andes virus, Sin Nombre virus, Crimean-Congo hemorrhagic fever virus, Influenza A virus, Influenza B virus. Influenza C virus, Hendra virus. Human parainfluenza virus 1, Human parainfluenza virus 2, Measles virus, Nipah virus, Mumps virus, Respiratory syncytial virus, Rinderpest virus, Canine distemper virus, Bunyamwera virus, Batai virus, Ngari virus, California encephalitis virus, Rift Valley fever virus, Human metapneumovirus, Respiratory syncytial virus, Rabies virus, Vesicular stomatitis virus, and Hepatitis D virus. Examples of (+)ssRNA viruses include Norovirus, Norwalk virus, Human coronavirus OC43, Human coronavirus strain 229E, Middle East respiratory syndrome coronavirus, Severe acute respiratory syndrome coronavirus, Severe acute respiratory syndrome coronavirus 2, Dengue virus, Hepatitis C virus, Japanese encephalitis virus, Kunjin virus, Powassan virus, Tick-borne encephalitis virus, West Nile virus, Yellow fever virus, Zika virus, Hepatitis E virus, Coxsackievirus A, Coxsackievirus B3, Coxsackievirus B4, Echovirus 1, Echovirus 6, Enterovirus 68, Enterovirus 70, Enterovirus 71, Human rhinovirus 14, Human rhinovirus 1B, Human rhinovirus 89, Human rhinovirus Δ16, Human rhinovirus Δ2, Parechovirus Δ3, Poliovirus, Saffold virus, Chikungunya virus, Ross River virus, Rubella virus, Sindbis virus and Eastern equine encephalitis. Examples of dsRNA viruses include Rotavirus. Examples of ssRNA-RT viruses include Human immunodeficiency virus 1 and Human immunodeficiency virus 2. Examples of ssDNA viruses include Parvovirus B19 and Canine Parvovirus. Examples of dsDNA viruses include Adenovirus, Cytomegalovirus, Epstein-Barr virus, Herpes simplex virus 1, Herpes simplex virus 2, Human herpesvirus 6, Human herpesvirus 7, Kaposi's sarcoma-associated herpesvirus, Varicella zoster virus, Human papillomavirus, BK virus, John Cunningham virus, Molluscum contagiosum virus, Vaccinia virus, Variola virus and Cowpox. Hepatitis B virus is an example of a virus of the dsDNA-RT group.

In one aspect, the invention provides a method wherein the cannabinoid compound or derivative thereof is administered with one or more additional therapeutic agents to said subject. For example, the additional therapeutic agent is an antiviral therapy.

Some embodiments of this invention relate to administering to the patient a therapeutically effective amount of a cannabinoid or derivative thereof or a compound of Formula I VIII, and one or more compounds selected from steroids, zinc supplements, nucleoside analogs, RNA-dependent RNA polymerase inhibitors, reverse transcriptase inhibitors, endocytosis inhibitors, autophagy inhibitors, antivirals, antibacterials, antiprotozoals, viral receptor antagonists, vitamin C, nucleotide analogs, protease inhibitors, membrane fusion inhibitors, antimalarials, ACE inhibitors, ACE2 inhibitors, ACE2 antibodies, recombinant ACE2, MASP-2 antibodies, C5-antibodies, immunomodulators, IL-1 inhibitors, IL-6 inhibitors, antibiotics, cardiovascular protective agents, anti-coagulants, statins, and pulmonary protective agents.

Some embodiments of this invention relate to administering to the patient a therapeutically effective amount of a cannabinoid or derivative thereof or a compound of Formula I VIII, and one or more compounds selected from dexamethasone, remdesivir, lopinavir, ritonavir, indinavir, atazanavir, boceprevir, darunavir, fosamprenavir, nelfinavir, saquinavir, simeprevir, telaprevir, tipranavir, favipiravir, umifenovir, azithromycin, tocilizumab, umifenovir, galidesivir, atorvastatin, and Pulmozyme.

Some embodiments of this invention relate to administering to the patient a therapeutically effective amount of a cannabinoid or derivative thereof or a compound of Formula I-VIII, and one or more compounds selected from Abacavir, Aciclovir, Acyclovir, Adefovir, Alisporivir, Amantadine, Amprenavir, Arbidol, Asunaprevir, Atazanavir, Baloxavir marboxil, Beclabuvir, Boceprevir, Brincidofovir, Brivudine, Cabotegravir, Cidofovir, Cobicistat, Combivir, Daclatasvir, Darunavir, Dasabuvir, Delavirdine, Didanosine, Docosanol, Dolutegravir, Doravirine, Edoxudine, Efavirenz, Elvitegravir, Emtricitabine, Enfuvirtide, Entecavir, Etravirine, Famciclovir, Favipiravir, Filociclovir, Fomivirsen, Fosamprenavir, Foscarnet, Galidesivir, Ganciclovir, Glecaprevir, Grazoprevir, Ibacitabine, Ibalizumab, Idoxuridine, Imiquimod, Imunovir, Indinavir, Interferon alfacon 1, Lamivudine, Laninamivir, octanoate, Letermovir, Lobucavir, Lopinavir, Loviride, Maraviroc, Maribavir, Methisazone, Moroxydine, Nelfinavir, Nevirapine, Nexavir, Nitazoxanide, N-methanocarbathymidine, Norvir, Oseltamivir, Oseltamivir phosphate, Paliviumab, Paritaprevir, Pegylated interferon, Pegylated interferon alfa 2a, Pegylated interferon alfa 2b, Penciclovir, Peramivir, Pibrentasvir, Pimodivir, Pleconaril, Podophyllotoxin, Raltegravir, Recombinant human interleukin-7, Remdesivir, Ribavirin, Rilpivirine, Rimantadine, Rintatolimod, Ritonavir, RSV IGIV, Saquinavir, Simeprevir, Sinecatechins, Sofosbuvir, Stavudine, Taribavirin, Tecovirimat, Telaprevir, Telbivudine, Tenofovir, Tenofovir alafenamide, Tenofovir disoproxil fumarate, Thymalfasin, Tipranavir, Trifluridine, Tromantadine, Umifenovir, Valaciclovir, Valacyclovir, Valganciclovir, Vaniprevir, VariZIG, Vicriviroc, Vidarabine, VZIG, Zalcitabine, Zanamivir, Zidovudine and Zinc gluconate.

Some embodiments of this invention relate to administering to the patient a therapeutically effective amount of a cannabinoid or derivative thereof or a compound of Formula I-VIII, and one or more agents selected from Actemra (tocilizumab), sotrovimab, baricitinib, and anti-SARS-CoV-2 monoclonal antibodies such as bamlanivimab, etesevimab, casirivimab, imdevimab, REGN-COV2 ((casirivimab with imdevimab), and regdanvimab and cortisteroids such as prednisone, methylprednisolone, or hydrocortisone.

Some embodiments of this invention relate to administering to a subject a therapeutically effective amount of a cannabinoid or derivative thereof or a compound of Formula I-VIII, or the pharmaceutically acceptable salt thereof, the tautomer thereof, the pharmaceutically acceptable salt of the tautomer, the stereoisomer of any of the foregoing, or the mixture thereof according to any one of the embodiments or a pharmaceutical composition of any of the embodiments.

The invention also relates to the use of the compounds of the present invention in maintenance treatment.

Since one aspect of the present invention contemplates the treatment of the disease/conditions with a combination of pharmaceutically active compounds that may be administered separately, the invention further relates to combining separate pharmaceutical compositions in kit form. The kit comprises two separate pharmaceutical compositions: the compound of the present invention, and a second pharmaceutical compound. The kit comprises a container for containing the separate compositions such as a divided bottle or a divided foil packet. Additional examples of containers include syringes, boxes and bags. Typically, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing physician or veterinarian.

Also provided are pharmaceutical compositions that include at least one pharmaceutically acceptable excipient, carrier or diluent and the compound or the pharmaceutically acceptable salt thereof, the tautomer thereof, the pharmaceutically acceptable salt of the tautomer, the stereoisomer of any of the foregoing, or the mixture thereof according to any one of the embodiments.

In one aspect, the invention provides a method wherein the cannabinoid compound or derivative thereof is a compound of Table 1.

TABLE 1
Naturally-Occurring Cannabinoid Compounds and Derivatives.

| | | | |
|---|---|---|---|
| Ex- | | | |
| ample | Compound | Synonym | Structure |

Cannabigerol-type (CBG)

| | | | |
|---|---|---|---|
| 1 | Cannabigerol | (E)-CBG-C5 | 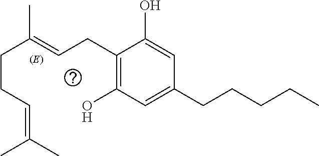 |
| 2 | Cannabigerol monomethyl ether | (E)-CBGM-C5 A | 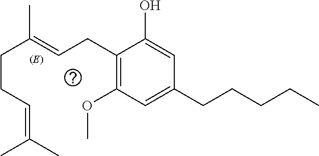 |
| 3 | Cannabinerolic acid A | (Z)-CBGA-C5 A | 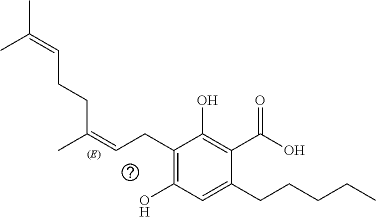 |
| 4 | Cannabigerovarin | (E)-CBGV-C3 | 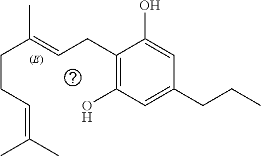 |
| 5 | Cannabigerolic acid A | (E)-CBGA-C5 A | 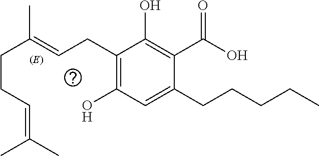 |
| 6 | Cannabigerolic acid A monomethyl ether | (E)-CBGAM-C5 A | 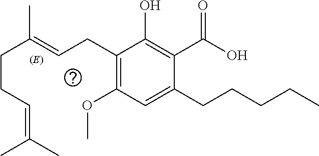 |
| 7 | Cannabigerovarinic acid A | (E)-CBGVA-C3 A | 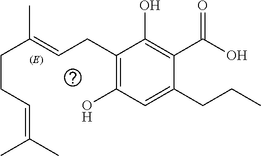 |

Cannabichromene-type (CBC)

| | | | |
|---|---|---|---|
| 8 | (±)-Cannabichromene | CBC-C5 | 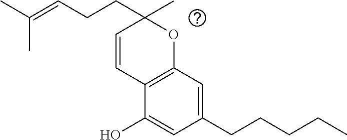 [SMILES: CCCCCc1cc(O)c2c(c1)OC(C)(CCC=C(C)C)C=C2] |
| 9 | (±)-Cannabichromenic acid A | CBCA-C5 A | 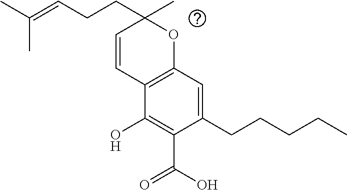 [SMILES: CCCCCc1cc2c(c(O)c1C(=O)O)C=CC(C)(CCC=C(C)C)O2] |
| 10 | (±)-Cannabivarichromene, (±)-Cannabichromevarin | CBCV-C3 | 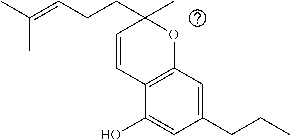 [SMILES: CCCc1cc(O)c2c(c1)OC(C)(CCC=C(C)C)C=C2] |
| 11 | (±)-Cannabichromevarinic acid A | CBCVA-C3 A | 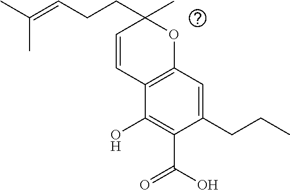 [SMILES: CCCc1cc2c(c(O)c1C(=O)O)C=CC(C)(CCC=C(C)C)O2] |

Cannabidiol-type (CBD)

| | | | |
|---|---|---|---|
| 12 | (−)-Cannabidiol | CBD-C5 | 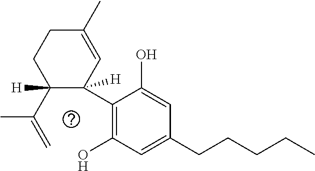 [SMILES: C=C(C)[C@@H]1CCC(C)=C[C@H]1c1c(O)cc(CCCCC)cc1O] |
| 13 | Cannabidiol monomethyl ether | CBDM-C5 | 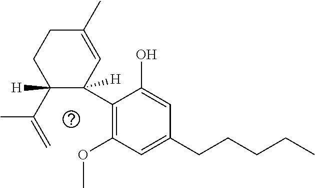 [SMILES: C=C(C)[C@@H]1CCC(C)=C[C@H]1c1c(O)cc(CCCCC)cc1OC] |
| 14 | Cannabidiol-C4 | CBD-C4 | 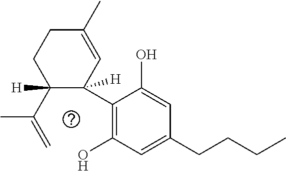 [SMILES: C=C(C)[C@@H]1CCC(C)=C[C@H]1c1c(O)cc(CCCC)cc1O] |
| 15 | (−)-Cannabidivarin | CBDV-C3 | 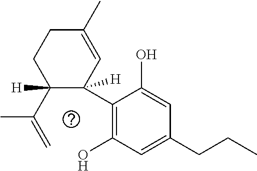 [SMILES: C=C(C)[C@@H]1CCC(C)=C[C@H]1c1c(O)cc(CCC)cc1O] |
| 16 | Cannabidiorcol | CBD-C1 | 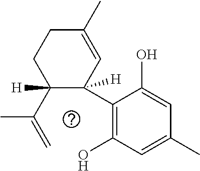 [SMILES: C=C(C)[C@@H]1CCC(C)=C[C@H]1c1c(O)cc(C)cc1O] |
| 17 | Cannabidiolic acid | CBDA-C5 | 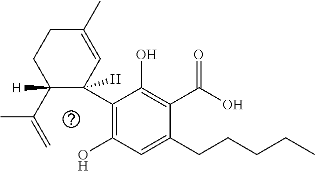 [SMILES: C=C(C)[C@@H]1CCC(C)=C[C@H]1c1c(O)cc(CCCCC)c(C(=O)O)c1O] |
| 18 | Cannabidivarinic acid | CBDVA-C3 | 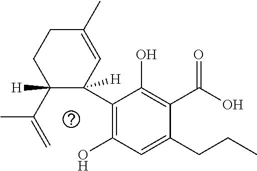 [SMILES: C=C(C)[C@@H]1CCC(C)=C[C@H]1c1c(O)cc(CCC)c(C(=O)O)c1O] |
| 19 | Cannabidiphorol | CBDP, CBD-C7 | 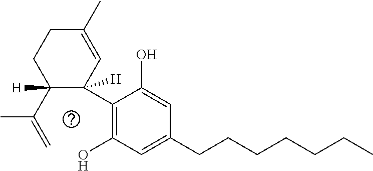 [SMILES: C=C(C)[C@@H]1CCC(C)=C[C@H]1c1c(O)cc(CCCCCCC)cc1O] |

Cannabinodiol-type (CBND)

| | | | |
|---|---|---|---|
| 20 | Cannabinodiol | CBND-C5 | 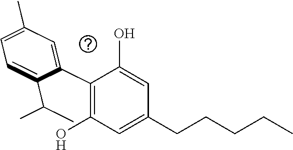 |
| 21 | Cannabinodivarin | CBND-C3 | 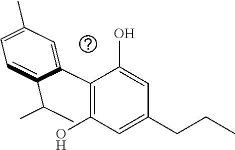 |

Tetrahydrocannabinol-type (THC)

| | | | |
|---|---|---|---|
| 22 | Δ9-Tetrahydrocannabinol | Δ9-THC-C5 | 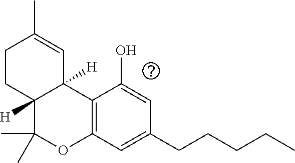 [SMILES: CCCCCc1cc(O)c2c(c1)OC(C)(C)[C@@H]1CCC(C)=C[C@@H]21] |
| 23 | Δ9-Tetrahydrocannabinol-C4 | Δ9-THC-C4 | 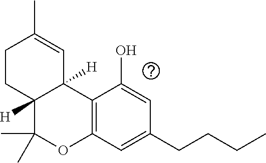 [SMILES: CCCCc1cc(O)c2c(c1)OC(C)(C)[C@@H]1CCC(C)=C[C@@H]21] |
| 24 | Δ9-Tetrahydrocannabivarin | Δ9-THCV-C3 | 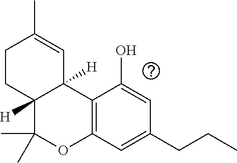 [SMILES: CCCc1cc(O)c2c(c1)OC(C)(C)[C@@H]1CCC(C)=C[C@@H]21] |
| 25 | Δ9-Tetrahydrocannabiorcol | Δ9-THCO-C1 | 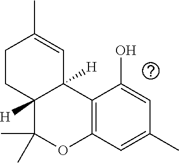 |
| 26 | Δ9-Tetrahydrocannabinolic acid A | Δ9-THCA-C5 A | 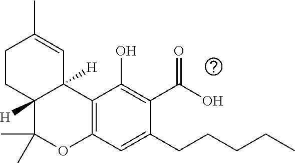 [SMILES: CCCCCc1cc2c(c(O)c1C(=O)O)[C@@H]1C=C(C)CC[C@H]1C(C)(C)O2] |
| 27 | Δ9-Tetrahydrocannabinolic acid B | Δ9-THCA-C5 B | 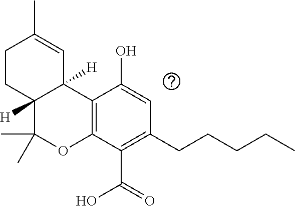 [SMILES: CCCCCc1cc(O)c2c(c1C(=O)O)OC(C)(C)[C@@H]1CCC(C)=C[C@@H]21] |
| 28 | Δ9-Tetrahydrocannabinolic acid-C4 A and/or B | Δ9-THCA-C4 A and/or B | 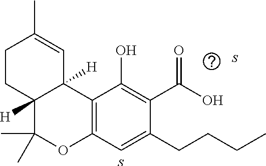 |
| 29 | Δ9-Tetrahydro- cannabivarinic acid A | Δ9-THCVA-C3 A | 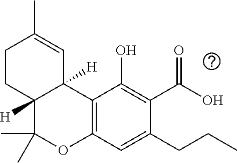 [SMILES: CCCc1cc2c(c(O)c1C(=O)O)[C@@H]1C=C(C)CC[C@H]1C(C)(C)O2] |
| 30 | Δ9-Tetrahydrocannabiorcolic acid A and/or B | Δ9-THCOA-C1 A and/or B | 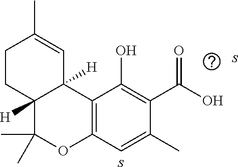 |
| 31 | (−)-Δ8-trans-(6aR,10aR)-Δ8- Tetrahydrocannabinol | Δ8-THC-C5 | 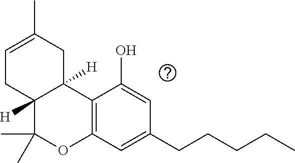 [SMILES: CCCCCc1cc(O)c2c(c1)OC(C)(C)[C@@H]1CC=C(C)C[C@@H]21] |
| 32 | (−)-Δ8-trans-(6aR,10aR)- Tetrahydrocannabinolic acid A | Δ8-THCA-C5 A | 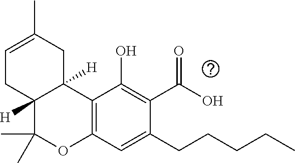 [SMILES: CCCCCc1cc2c(c(O)c1C(=O)O)[C@@H]1CC(C)=CC[C@H]1C(C)(C)O2] |
| 33 | (−)-(6aS,10aR)-Δ9- Tetrahydrocannabinol | (−)-cis-Δ9-THC-C5 | 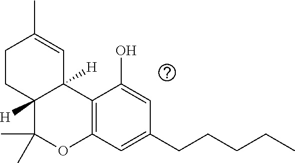 [SMILES: CCCCCc1cc(O)c2c(c1)OC(C)(C)[C@@H]1CCC(C)=C[C@@H]21] |
| 34 | Δ9-Tetrahydrocannabiphorol | Δ9-THCP, Δ9-THC-C7 | 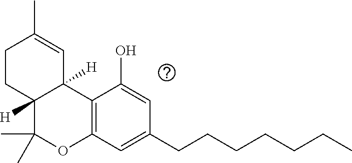 [SMILES: CCCCCCCc1cc(O)c2c(c1)OC(C)(C)[C@@H]1CCC(C)=C[C@@H]21] |

Cannabinol-type (CBN)

| | | | |
|---|---|---|---|
| 35 | Cannabinol | CBN-C5 | 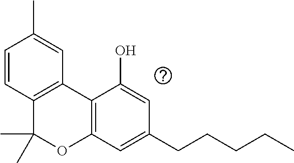 [SMILES: CCCCCc1cc(O)c2c(c1)OC(C)(C)c1ccc(C)cc1-2] |
| 36 | Cannabinol-C4 | CBN-C4 | 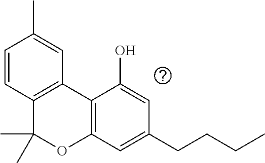 [SMILES: CCCCc1cc(O)c2c(c1)OC(C)(C)c1ccc(C)cc1-2] |
| 37 | Cannabivarin | CBN-C3 | 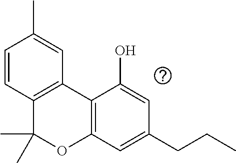 [SMILES: CCCc1cc(O)c2c(c1)OC(C)(C)c1ccc(C)cc1-2] |
| 38 | Cannabinol-C2 | CBN-C2 | 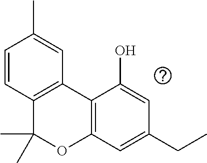 [SMILES: CCc1cc(O)c2c(c1)OC(C)(C)c1ccc(C)cc1-2] |
| 39 | Cannabiorcol | CBN-C1 | 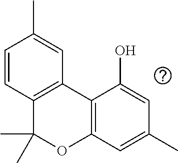 |
| 40 | Cannabinolic acid A | CBNA-C5 A | 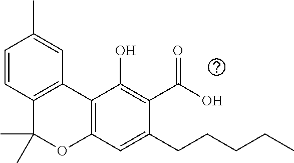 [SMILES: CCCCCc1cc2c(c(O)c1C(=O)O)-c1cc(C)ccc1C(C)(C)O2] |
| 41 | Cannabinol methyl ether | CBNM-C5 | 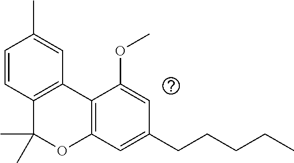 [SMILES: CCCCCc1cc(OC)c2c(c1)OC(C)(C)c1ccc(C)cc1-2] |

Cannabitriol-type (CBT)

| | | | |
|---|---|---|---|
| 42 | (−)-(9R,10R)-trans-Cannabitriol | (−)-trans-CBT-C5 | 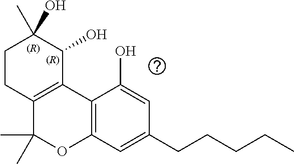 |
| 43 | (+)-(9S,10S)-Cannabitriol | (+)-trans-CBT-C5 | 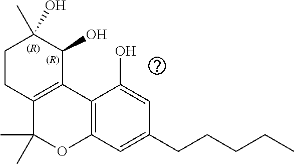 |
| 44 | (+)-(9R,10S/9S,10R)-Cannabitriol | (+)-cis-CBT-C5 | 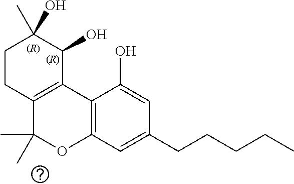 |
| 45 | (−)-(9R,10R)-trans-10-O-Ethyl- cannabitriol | (−)-trans-CBT- OEt-C5 | 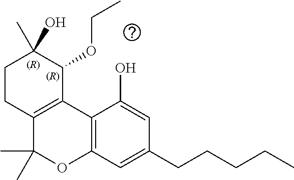 |
| 46 | (±)-(9R,10R/9S,10S)- Cannabitriol-C3 | (±)-trans-CBT-C3 | 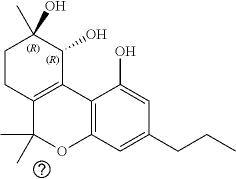 |
| 47 | 8,9-Dihydroxy-Δ6a(10a)- tetrahydrocannabinol | 8,9-Di-OH- CBT-C5 | 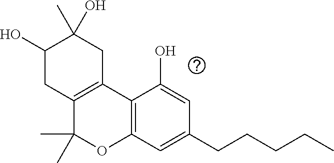 [SMILES: CCCCCc1cc(O)c2c(c1)OC(C)(C)C1=C2CC(C)(O)C(O)C1] |
| 48 | Cannabidiolic acid A cannabitriol ester | CBDA-C5 9-OH- CBT-C5 ester | 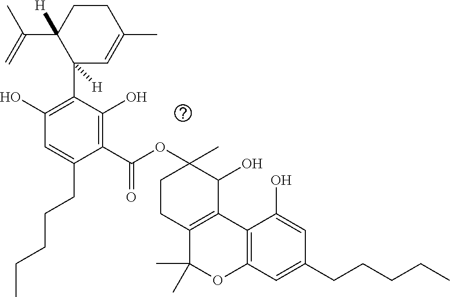 [SMILES: C=C(C)[C@@H]1CCC(C)=C[C@H]1c1c(O)cc(CCCCC)c(C(=O)OC2(C)CCC3=C(c4c(O)cc(CCCCC)cc4OC3(C)C)C2O)c1O] |
| 49 | (−)-(6aR,9S,10S,10aR)-9,10- Dihydroxy-hexahydrocannabinol, Cannabiripsol | Cannabiripsol-C5 | 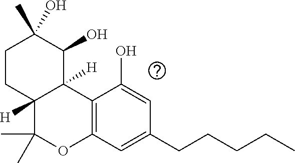 [SMILES: CCCCCc1cc(O)c2c(c1)OC(C)(C)[C@@H]1CC[C@](C)(O)[C@@H](O)[C@@H]21] |
| 50 | (−)-6a,7,10a-Trihydroxy-Δ9- tetrahydrocannabinol | (−)-Cannabitetrol | 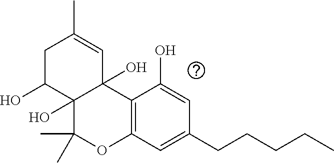 [SMILES: CCCCCc1cc(O)c2c(c1)OC(C)(C)C1(O)C(O)CC(C)=CC21O] |
| 51 | 10-Oxo-Δ6a(10a)- tetrahydrocannabinol | OTHC | 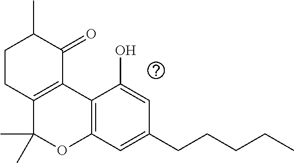 [SMILES: CCCCCc1cc(O)c2c(c1)OC(C)(C)C1=C2C(=O)C(C)CC1] |

Cannabielsoin-type (CBE)

| | | | |
|---|---|---|---|
| 52 | (5aS,6S,9R,9aR)-Cannabielsoin | CBE-C5 | 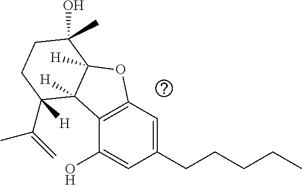 |
| 53 | (5aS,6S,9R,9aR)-C3-Cannabielsoin | CBE-C3 | 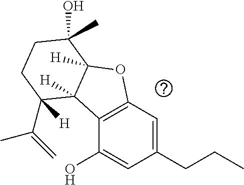 |
| 54 | (5aS,6S,9R,9aR)- Cannabielsoic acid A | CBEA-C5 A | 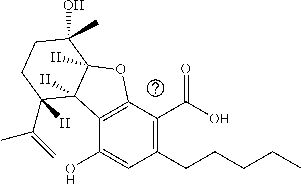 |
| 55 | (5aS,6S,9R,9aR)- Cannabielsoic acid B | CBEA-C5 B | 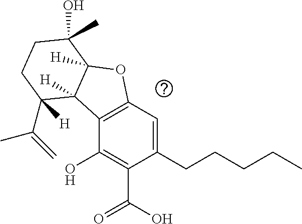 |
| 56 | (5aS,6S,9R,9aR)-C3- Cannabielsoic acid B | CBEA-C3 B | 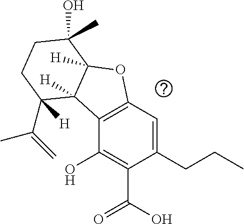 |
| 57 | Cannabiglendol-C3 | OH-iso-HHCV-C3 | 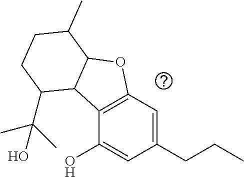 [SMILES: CCCc1cc(O)c2c(c1)OC1C(C)CCC(C(C)(C)O)C21] |
| 58 | Dehydrocannabifuran | DCBF-C5 | 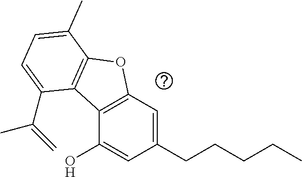 [SMILES: C=C(C)c1ccc(C)c2oc3cc(CCCCC)cc(O)c3c12] |
| 59 | Cannabifuran | CBF-C5 | 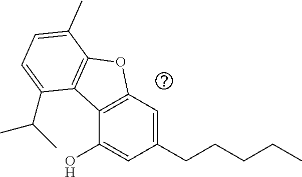 [SMILES: CCCCCc1cc(O)c2c(c1)oc1c(C)ccc(C(C)C)c12] |

Isocannabinoids

| | | |
|---|---|---|
| 60 | (−)-Δ7-trans-(1R,3R,6R)- Isotetrahydrocannabinol | 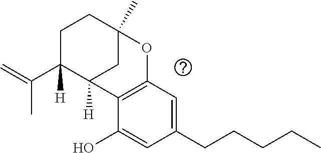 [SMILES: C=C(C)[C@@H]1CC[C@]2(C)C[C@H]1c1c(O)cc(CCCCC)cc1O2] |
| 61 | (±)-Δ7-1,2-cis- (1R,3R,6S/1S,3S,6R)- Isotetrahydrocannabivarin | 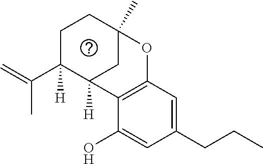 [SMILES: C=C(C)[C@H]1CC[C@]2(C)C[C@H]1c1c(O)cc(CCC)cc1O2] |
| 62 | (−)-Δ7-trans-(1R,3R,6R)- Isotetrahydrocannabivarin | 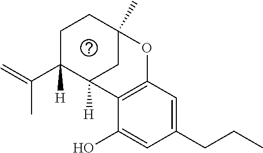 [SMILES: C=C(C)[C@@H]1CC[C@]2(C)C[C@H]1c1c(O)cc(CCC)cc1O2] |

Cannabicyclol-type (CBL)

| | | | |
|---|---|---|---|
| 63 | (±)-(1aS,3aR,8bR,8cR)- Cannabicyclol | CBL-C5 | 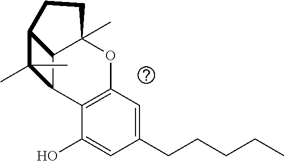 |
| 64 | (±)-(1aS,3aR,8bR,8cR)- Cannabicyclolic acid A | CBLA-C5 A | 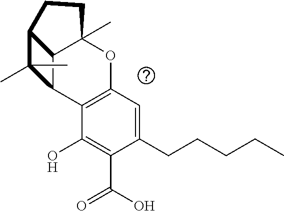 |
| 65 | (±)-(1aS,3aR,8bR,8cR)- Cannabicyclovarin | CBLV-C3 | 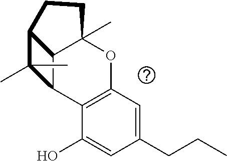 |

Cannabicitran-type (CBT)

| | | | |
|---|---|---|---|
| 66 | Cannabicitran | CBT-C5 | 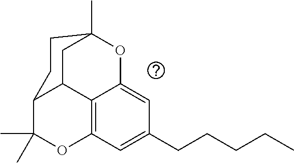 [SMILES: CCCCCc1cc2c3c(c1)OC(C)(C)C1CCC(C)(CC31)O2] |

Cannabichromanone-type (CBCN)

| | | | |
|---|---|---|---|
| 67 | Cannabichromanone | CBCN-C5 | 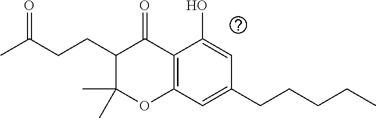 [SMILES: CCCCCc1cc(O)c2c(c1)OC(C)(C)C(CCC(C)=O)C2=O] |
| 68 | Cannabichromanone-C3 | CBCN-C3 | 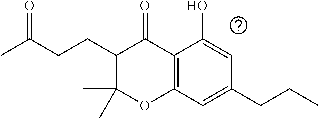 [SMILES: CCCc1cc(O)c2c(c1)OC(C)(C)C(CCC(C)=O)C2=O] |
| 69 | Cannabicoumaronone | CBCON-C5 | 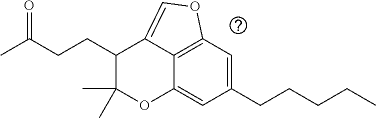 [SMILES: CCCCCc1cc2c3c(coc3c1)C(CCC(C)=O)C(C)(C)O2] |

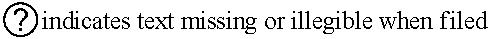

in one aspect, the invention provides a method wherein the cannabinoid compound or derivative thereof is a compound of Table 2.

TABLE 2
Synthetic Cannabinoid Compounds.

| Example | Synonym | Compound |
|---|---|---|
| 70 | A-836339 | [N(Z)]-N-[3-(2-methoxyethyl)-4,5-dimethyl-2(3H)- |
| | | thiazolylidene]-2,2,3,3-tetramethyl- |
| | | cyclopropanecarboxamide |
| 71 | AM3102 | N-[(1R)-2-hydroxy-1-methylethyl-9Z-octadecenamide |
| 72 | CB-13 | 1-naphthalenyl[4-(pentoxy)-1-naphthalenyl]- |
| | | methanone |
| 73 | CB-25 | N-cyclopropyl-11-(3-hydroxy-5-pentylphenoxy)- |
| | | undecanamide |
| 74 | CB-52 | N-cyclopropyl-11-(2-hexyl-5-hydroxyphenoxy)- |
| | | undecanamide |
| 75 | CB-86 | N-cyclopropyl-8-[3-(1,1-dimethylheptyl)-5- |
| | | hydroxyphenoxy]-octanamide |
| 76 | (+)-CP 47,497 | 2[(1R,3S)-3-hydroxycyclohexyl]-5-(2-methyloctan-2- |
| | | yl)phenol |
| 77 | (−)-CP 47,497 | 2[(1S,3R)-3-hydroxycyclohexyl]-5-(2-methyloctan-2- |
| | | yl)phenol |
| 78 | (+/−)-CP 47,497 | rel-5-(1,1-dimethylheptyl)-2-[(1R,3S)-3- |
| | | hydroxycyclohexyl]-phenol |
| 79 | (+/−)3-epi CP 47,497 | rel-2[(1S,3S)-3-hydroxycyclohexyl)]-5-(2-methyloctan- |
| | | 2-yl)phenol |
| 80 | CP 47,497-C6-homolog | cis-5-(1,1-dimethylhexyl)-2-(3-hydroxycyclohexyl)- |
| | | phenol |
| 81 | (+/−)-CP 47,497-C8- | rel-5-(1,1-dimethyloctyl)-2-[(1R,3S)-3- |
| | homolog | hydroxycyclohexyl]-phenol |
| 82 | (+/−)3-epi CP 47,497-C8- | rel-2-[(1S,3S)-3-hydroxycyclohexyl)]-5-(2-methylnonan- |
| | homolog | 2-yl)phenol |
| 83 | CP 47,497-C9-homolog | cis-5-(1,1-dimethylnonyl)-2-(3-hydroxycyclohexyl)- |
| | | phenol |
| 84 | CP 47,497-para-quinone | 3′R-hydroxy-4-(2-methyloctan-2-yl)-[1,1′S- |
| | analog | bi(cyclohexane)]-3,6-diene-2,5-dione |
| 85 | (+)-CP 55,940 | 2-((1S,2S,5S)-5-hydroxy-2-(3- |
| | | hydroxypropyl)cyclohexyl)-5-(2-methyloctan-2- |
| | | yl)phenol |
| 86 | (−)-CP 55,940 | 5-(1,1-dimethylheptyl)-2-[(1R,2R,5R)-5-hydroxy-2-(3- |
| | | hydroxypropyl)cyclohexyl]-phenol |
| 87 | (+/−)CP 55,940 | rel-5-(1,1-dimethylheptyl)-2-[(1R,2R,5R)-5-hydroxy-2- |
| | | (3-hydroxypropyl)cyclohexyl]-phenol |
| 88 | (+/−)5-epi CP 55,940 | rel-2-((1R,2R,5S)-5-hydroxy-2-(3- |
| | | hydroxypropyl)cyclohexyl)-5-(2-methyloctan-2- |
| | | yl)phenol |
| 89 | Flurazepam | 7-chloro-1-[2-(diethylamino)ethyl]-5-(2-fluorophenyl)- |
| | | 1,3-dihydro-2H-1,4-benzodiazepin-2-one |
| 90 | HU-210 | 3-(1,1′-dimethylheptyl)-6aR,7,10,10aR-tetrahydro-1- |
| | | hydroxy-6,6-dimethyl-6H-dibenzo[b,d]pyran-9-methanol |
| | | 3-(1,1-dimethylheptyl)-6aS,7,10,10aS-tetrahydro-1- |
| 91 | HU-211 | hydroxy-6,6-dimethyl-6H-dibenzo[b,d]pyran-9- |
| | | methanol |
| 92 | HU-239, Ajulemic acid | (6aR,10aR)-1-hydroxy-6,6-dimethyl-3-(2-methyloctan- |
| | | 2-yl)-6a,7,10,10a-tetrahydrobenzo[c]chromene-9- |
| | | carboxylic acid |
| 93 | HU-308 | 4-[4-(1,1-dimethylheptyl)-2,6-dimethoxyphenyl]-6,6- |
| | | dimethyl-bicyclo[3.1.1]hept-2-ene-2-methanol |
| 94 | HU-320 | (3S,4S)-3-[2,6-Dihydroxy-4-(2-methyl-2- |
| | | octanyl)phenyl]-4-isopropenyl-1-cyclohexene-1- |
| | | carboxylic acid |
| 95 | HU-331 | 3-hydroxy-2-[(1R,6R)-3-methyl-6-(1-methylethenyl)-2- |
| | | cyclohexen-1-yl]-5-pentyl-2,5-cyclohexadiene-1,4- |
| | | dione |
| 96 | HU-336 | (6aR,10aR)-6,6,9-Trimethyl-3-pentyl-6a,7,10,10a- |
| | | tetrahydro-1H-dibenzo[b,d]pyran-1,4(6H)-dione |
| 97 | HU-243 | ((6aR,8S,9S,10aR)-9-(Hydroxymethyl)-6,6-dimethyl-3- |
| | | (2-methyloctan-2-yl)-8,9-ditritio-7,8,10,10a-tetrahydro- |
| | | 6aH-benzo[c]chromen-1-ol). |
| 98 | HU-345 | 6,6,9-Trimethyl-3-pentyl-1H-benzo[c]chromene- |
| | | 1,4(6H)-dione |
| 99 | JP104 | 3′-carbamoyl-biphenyl-3-yl-undecynecarbamate |
| 100 | JWH 176 | 1-[(E)-(3-pentyl-1H-inden-1-ylidene)methyl]- |
| | | naphthalene |
| 101 | JWH 133 | 3-(1,1-dimethylbutyl)-6aR,7,10,10aR-tetrahydro-6,6,9- |
| | | trimethyl-6H-dibenzo[b,d]pyran |
| 102 | KLS-13019 | 1-(3-(((1′R,2′R)-2,6-dihydroxy-5′-methyl-2′-(prop-1-en- |
| | | 2-yl)-1′,2′,3′,4'-tetrahydro-[1,1′-biphenyl]-4- |
| | | yl)methyl)azetidin-1-yl)ethan-1-one |
| 103 | KM 233 | 6aR,7,10,10aR-tetrahydro-6,6,9-trimethyl-3-(1-methyl- |
| | | 1-phenylethyl)-6H-dibenzo[b,d]pyran-1-ol |
| 104 | 3,4-MDMA methylene | N,β-dimethyl-1,3-benzodioxole-5-propanamine, |
| | homolog (hydrochloride) | monohydrochloride |
| 105 | 4-MeOPP | 1-(4-Methoxyphenyl) piperazine (hydrochloride) |
| 106 | Nabilone, Cesamet | (6aR,10aR)-1-hydroxy-6,6-dimethyl-3-(2-methyloctan- |
| | | 2-yl)-6H,6aH,7H,8H,9H,10H,10aH- |
| | | benzo[c]isochromen-9-one |
| 107 | PSB-SB1202 | 5-methoxy-3-[(2-methoxyphenyl)methyl]-7-pentyl-2H-1- |
| | | benzopyran-2-one |
| 108 | LY2183240 | 5-([1,1′-biphenyl]-4-ylmethyl)-N,N-dimethyl-1H- |
| | | tetrazole-1-carboxamide |
| 109 | LY2183240 2′-isomer | 5-([1,1′-biphenyl]-4-ylmethyl)-N,N-dimethyl-2H- |
| | | tetrazole-2-carboxamide |
| 110 | URB602 | N-[1,1′-biphenyl]-3-yl-carbamic acid, cyclohexyl ester |
| 111 | A-796260 | [1-[2-(4-morpholinyl)ethyl]-1H-indol-3-yl](2,2,3,3- |
| | | tetramethylcyclopropyl)-methanone |
| 112 | A-834735 | [1-[(tetrahydro-2H-pyran-4-yl)methyl]-1H-indol-3- |
| | | yl](2,2,3,3-tetramethylcyclopropyl)-methanone |
| 113 | AB-005 | [1-[(1-methyl-2-piperidinyl)methyl]-1H-indol-3- |
| | | yl](2,2,3,3-tetramethylcyclopropyl)-methanone |
| 114 | AB-005 azepane isomer | (1-(1-methylazepan-3-yl)-1H-indol-3-yl)(2,2,3,3- |
| | | tetramethylcyclopropyl)methanone |
| 115 | AB-BICA | N-[(1S)-1-(aminocarbonyl)-2-methylpropyl]-1- |
| | | (phenylmethyl)-1H-indole-3-carboxamide |
| 116 | 5-fluoro ABICA | N-[(1S)-1-(aminocarbonyl)-2-methylpropyl]-1-(5- |
| | | fluoropentyl)-1H-indole-3-carboxamide |
| 117 | AB-CHMICA | (S)-N-(1-amino-3-methyl-1-oxobutan-2-yl)-1- |
| | | (cyclohexylmethyl)-1H-indole-3-carboxamide |
| 118 | AB-CHMINACA | N-[(1S)-1-(aminocarbonyl)-2-methylpropyl]-1- |
| | | (cyclohexylmethyl)-1H-indazole-3-carboxamide |
| 119 | AB-CHMINACA 2′-indazole | (S)-N-(1-amino-3-methyl-1-oxobutan-2-yl)-2- |
| | isomer | (cyclohexylmethyl)-2H-indazole-3-carboxamide |
| 120 | 5,3-AB-CHMFUPPYCA | (S)-N-(1-amino-3-methyl-1-oxobutan-2-yl)-1- |
| | | (cyclohexylmethyl)-5-(4-fluorophenyl)-1H-pyrazole-3- |
| | | carboxamide |
| 121 | AB-FUBICA | N-[(1S)-1-(aminocarbonyl)-2-methylpropyl]-1-[(4- |
| | | fluorophenyl)methyl]-1H-indole-3-carboxamide |
| 122 | AB-FUBINACA | N-[(1S)-1-(aminocarbonyl)-2-methylpropyl]-1-[(4- |
| | | fluorophenyl)methyl]-1H-indazole-3-carboxamide |
| 123 | AB-FUBINACA 2-F-benzyl | N-[(1S)-1-(aminocarbonyl)-2-methylpropyl]-1-[(2- |
| | isomer | fluorophenyl)methyl]-1H-indazole-3-carboxamide |
| 124 | AB-FUBINACA 3-F-benzyl | N-[(1S)-1-(aminocarbonyl)-2-methylpropyl]-1-[(3- |
| | isomer | fluorophenyl)methyl]-1H-indazole-3-carboxamide |
| 125 | AB-FUBINACA isomer 1 | N-(1-amino-1-oxopentan-2-yl)-1-(4-fluorobenzyl)-1H- |
| | | indazole-3-carboxamide |
| 126 | AB-FUBINACA isomer 2 | N-(1-amino-2-methyl-1-oxobutan-2-yl)-1-(4- |
| | | fluorobenzyl)-1H-indazole-3-carboxamide |
| 127 | AB-FUBINACA isomer 5 | N-(1-amino-1-oxobutan-2-yl)-1-(4-fluorobenzyl)-N- |
| | | methyl-1H-indazole-3-carboxamide |
| 128 | 5-fluoro-3,5-AB- | (S)-N-(1-amino-3-methyl-1-oxobutan-2-yl)-1-(5- |
| | PFUPPYCA | fluoropentyl)-3-(4-fluorophenyl)-1H-pyrazole-5- |
| | | carboxamide |
| 129 | AB-PINACA | (S)-N-(1-amino-3-methyl-1-oxobutan-2-yl)-1-pentyl-1H- |
| | | indazole-3-carboxamide |
| 130 | 5-chloro AB-PINACA | N-[(1S)-1-(aminocarbonyl)-2-methylpropyl]-1-(5- |
| | | chloropentyl)-1H-indazole-3-carboxamide |
| 131 | 5-fluoro AB-PINACA | N-[(1S)-1-(aminocarbonyl)-2-methylpropyl]-1-(5- |
| | | fluoropentyl)-1H-indazole-3-carboxamide |
| 132 | AB-PINACA N-(2- | N-(1-amino-3-methyl-1-oxobutan-2-yl)-1-(2- |
| | fluoropentyl) isomer | fluoropentyl)-1H-indazole-3-carboxamide |
| 133 | AB-PINACA N-(3- | N-(1-amino-3-methyl-1-oxobutan-2-yl)-1-(3- |
| | fluoropentyl) isomer | fluoropentyl)-1H-indazole-3-carboxamide |
| 134 | AB-PINACA N-(4- | N-(1-amino-3-methyl-1-oxobutan-2-yl)-1-(4- |
| | fluoropentyl) isomer | fluoropentyl)-1H-indazole-3-carboxamide |
| 135 | ADB-BICA | N-(1-amino-3,3-dimethyl-1-oxobutan-2-yl)-1-benzyl-1H- |
| | | indole-3-carboxamide |
| 136 | ADB-BINACA | N-(1-amino-3,3-dimethyl-1-oxobutan-2-yl)-1-benzyl-1H- |
| | | indazole-3-carboxamide |
| 137 | ADB-FUBINACA | N-[1-(aminocarbonyl)-2,2-dimethylpropyl]-1-[(4- |
| | | fluorophenyl)methyl]-1H-indazole-3-carboxamide |
| 138 | 4-fluoro ADB | methyl (2S)-2-(1-(4-fluoropentyl)-1H-indazole-3- |
| | | carboxamido)-3,3-dimethylbutanoate |
| 139 | 5-fluoro ADB | N-[[1-(5-fluoropentyl)-1H-indazol-3-yl]carbonyl]-3- |
| | | methyl-D-valine, methyl ester |
| 140 | 5-fluoro ADB-PINACA | N-[1-(aminocarbonyl)-2,2-dimethylpropyl]-1-(5- |
| | | fluoropentyl)-1H-indazole-3-carboxamide |
| 141 | 5-fluoro ADB-PINACA | N-((2S,3S)-1-amino-3-methyl-1-oxopentan-2-yl)-1-(5- |
| | isomer 2 | fluoropentyl)-1H-indazole-3-carboxamide |
| 142 | 5-fluoro-2-ADB-PINACA | N-(1-amino-3S-methyl-1-oxopentan-2S-yl)-2-(5- |
| | isomer 2 | fluoropenty!)-2H-indazole-3-carboxamide |
| 143 | 5-fluoro-3,5-ADB- | N-[1-(aminocarbonyl)-2,2-dimethylpropyl]-1-(5- |
| | PFUPPYCA | fluoropentyl)-3-(4-fluorophenyl)-1H-pyrazole-5- |
| | | carboxamide |
| 144 | ADBICA | N-(1-amino-3,3-dimethyl-1-oxobutan-2-yl)-1-pentyl-1H- |
| | | indole-3-carboxamide |
| 145 | 5-fluoro ADBICA | N-[1-(aminocarbonyl)-2,2-dimethylpropyl]-1-(5- |
| | | fluoropentyl)-1H-indole-3-carboxamide |
| 146 | ADB-PINACA | N-[1-(aminocarbonyl)-2,2-dimethylpropyl]-1-pentyl-1H- |
| | | indazole-3-carboxamide |
| 147 | ADB-PINACA isomer 1 | N-(1-amino-2,3-dimethyl-1-oxobutan-2-yl)-1-pentyl-1H- |
| | | indazole-3-carboxamide |
| 148 | ADB-PINACA isomer 2 | N-((2S,3S)-1-amino-3-methyl-1-oxopentan-2-yl)-1- |
| | | pentyl-1H-indazole-3-carboxamide |
| 149 | ADB-PINACA isomer 3 | (S)-N-(1-amino-1-oxohexan-2-yl)-1-pentyl-1H- |
| | | indazole-3-carboxamide |
| 150 | ADB-PINACA isomer 4 | (S)-N-(1-amino-4-methyl-1-oxopentan-2-yl)-1-pentyl- |
| | | 1H-indazole-3-carboxamide |
| 151 | 5-fluoro AEB | ethyl (1-(5-fluoropentyl)-1H-indazole-3-carbonyl)-L- |
| | | valinate |
| 152 | AKB48 N-(4-F-benzyl) | N-((3s,5s,7s)-adamantan-1-yl)-1-(4-fluorobenzyl)-1H- |
| | analog | indazole-3-carboxamide |
| 153 | AKB48 N-(5-fluoropentyl) | N-((3s,5s,7s)-adamantan-1-yl)-1-(5-fluoropentyl)-1H- |
| | analog | indazole-3-carboxamide |
| 154 | 5-chloro AKB48 | N-((3s,5s,7s)-adamantan-1-yl)-1-(5-chloropentyl)-1H- |
| | | indazole-3-carboxamide |
| 155 | AM630 | [6-iodo-2-methyl-1-[2-(4-morpholinyl)ethyl]-1H-indol-3- |
| | | yl](4-methoxyphenyl)-methanone |
| 156 | AM679 | (2-iodophenyl)(1-pentyl-1H-indol-3-yl)-methanone |
| 157 | AM694 | [1-(5-fluoropentyl)-1H-indol-3-yl](2-iodophenyl)- |
| | | methanone |
| 158 | AM694 3-iodo isomer | (1-(5-fluoropentyl)-1H-indol-3-yl)(3- |
| | | iodophenyl)methanone |
| 159 | AM694 4-iodo isomer | (1-(5-fluoropentyl)-1H-indol-3-yl)(4- |
| | | iodophenyl)methanone |
| 160 | AM1220 | [1-[(1-methyl-2-piperidinyl)methyl]-1H-indol-3-yl]-1- |
| | | naphthalenyl-methanone |
| 161 | AM1220 azepane isomer | (1-((1-methylazepan-3-yl)methyl)-1H-indol-3- |
| | | yl) (naphthalen-1-yl)methanone |
| 162 | AM1235 | [1-(5-fluoropentyl)-6-nitro-1H-indol-3-yl]-1-naphthalenyl- |
| | | methanone |
| 163 | AM1241 | (2-iodo-5-nitrophenyl)-(1-(1-methylpiperidin-2- |
| | | ylmethyl)-1H-indol-3-yl)methanone |
| 164 | (R)-AM1241 | (2-iodo-5-nitrophenyl)[1-[[(2R)-1-methyl-2- |
| | | piperidinyl]methyl]-1H-indol-3-yl]methanone |
| 165 | AM1248 | [1-[(1-methyl-2-piperidinyl)methyl]-1H-indol-3- |
| | | yl]tricyclo[3.3.1.13,7]dec-1-yl-methanone |
| 166 | AM1248 azepane isomer | (3,5,7)-adamantan-1-yl(1-(1-methylazepan-3-yl)-1H- |
| | | indol-3-yl)methanone |
| 167 | AM2201 | [1-(5-fluoropentyl)-1H-indol-3-yl]-1-naphthalenyl- |
| | | methanone |
| 168 | AM2201 2′-naphthyl isomer | (1-(5-fluoropentyl)-1H-indol-3-yl)(naphthalen-2- |
| | | yl)methanone |
| 169 | AM2201 8-quinolinyl | 1-(5-fluoropentyl)-N-(quinolin-8-yl)-1H-indole-3- |
| | carboxamide | carboxamide |
| 170 | AM2201 benzimidazole | (1-(5-fluoropentyl)-1H-benzo[d]imidazol-2- |
| | analog | yl) (naphthalen-1-yl)methanone |
| 171 | AM2201 N-(2-F-pentyl) | (1-(2-fluoropentyl)-1H-indol-3-yl)(naphthalen-1- |
| | isomer | yl)methanone |
| 172 | AM2201 N-(3-Cl-pentyl) | (1-(3-chloropentyl)-1H-indol-3-yl)(naphthalen-1- |
| | isomer | yl) methanone |
| 173 | AM2201 N-(3-fluoropentyl) | (1-(3-fluoropentyl)-1H-indol-3-yl)(naphthalen-1- |
| | isomer | yl)methanone |
| 174 | AM2201 N-(4-fluoropentyl) | (1-(4-fluoropentyl)-1H-indol-3-yl)(naphthalen-1- |
| | isomer | yl)methanone |
| 175 | AM2232 | 3-(1-naphthalenylcarbonyl)-1H-indole-1-pentanenitrile |
| 176 | AM2233 | (2-iodophenyl)[1-[(1-methyl-2-piperidinyl)methyl]-1H- |
| | | indol-3-yl]-methanone |
| 177 | AM2233 azepane isomer | (2-iodophenyl)(1-(1-methylazepan-3-yl)-1H-indol-3- |
| | | yl)methanone |
| 178 | AMB | N-[(1-pentyl-1H-indazol-3-yl)carbonyl]-L-valine, methyl |
| | | ester |
| 179 | 3-fluoro AMB | methyl (1-(3-fluoropentyl)-1H-indazole-3-carbonyl)-L- |
| | | valinate |
| 180 | 4-fluoro AMB | methyl (1-(4-fluoropentyl)-1H-indazole-3-carbonyl)-L- |
| | | valinate |
| 181 | 5-fluoro AMB | N-[[1-(5-fluoropentyl)-1H-indazol-3-yl]carbonyl]-L- |
| | | valine, methyl ester |
| 182 | APINAC | (3s,5s,7s)-adamantan-1-yl 1-pentyl-1H-indazole-3- |
| | | carboxylate |
| 183 | 5-fluoro APINAC | (3s,5s,7s)-adamantan-1-yl 1-(5-fluoropentyl)-1H- |
| | | indazole-3-carboxylate |
| 184 | APP-CHMINACA | N-[(1S)-2-amino-2-oxo-1-(phenylmethyl)ethyl]-1- |
| | | (cyclohexylmethyl)-1H-indazole-3-carboxamide |
| 185 | APP-FUBINACA | N-[(1S)-2-amino-2-oxo-1-(phenylmethyl)ethyl]-1-[(4- |
| | | fluorophenyl)methyl]-1H-indazole-3-carboxamide |
| 186 | APP-PICA | (S)-N-(1-amino-1-oxo-3-phenylpropan-2-yl)-1-pentyl- |
| | | 1H-indole-3-carboxamide |
| 187 | ATHPINACA isomer 1 | 1-[(tetrahydro-2H-pyran-4-yl)methyl]-N- |
| | | tricyclo[3.3.1.1^{3,7}]dec-1-yl-1H-indazole-3-carboxamide |
| 188 | ATHPINACA isomer 2 | N-(adamantan-2-yl)-1-((tetrahydro-2H-pyran-4- |
| | | yl)methyl)-1H-indazole-3-carboxamide |
| 189 | Azidoindolene 1 | 2,2,3,3-tetramethyl-cyclopropanecarboxylic acid (2Z)-2- |
| | | [1-(5-fluoropentyl)-1,2-dihydro-2-oxo-3H-indol-3- |
| | | ylidene]hydrazide |
| 190 | BB-22 | 1-(cyclohexylmethyl)-8-quinolinyl ester-1H-indole-3- |
| | | carboxylic acid |
| 191 | BB-22 3-HO-quinoline | Quinolin-3-yl 1-(cyclohexylmethyl)-1H-indole-3- |
| | isomer | carboxylate |
| 192 | BB-22 4-HO-isoquinoline | isoquinolin-4-yl 1-(cyclohexylmethyl)-1H-indole-3- |
| | isomer | carboxylate |
| 193 | BB-22 4-HO-quinoline | quinolin-4-yl 1-(cyclohexylmethyl)-1H-indole-3- |
| | isomer | carboxylate |
| 194 | BB-22 5-HO-isoquinoline | isoquinolin-5-yl 1-(cyclohexylmethyl)-1H-indole-3- |
| | isomer | carboxylate |
| 195 | BB-22 5-HO-quinoline | quinolin-5-yl 1-(cyclohexylmethyl)-1H-indole-3- |
| | isomer | carboxylate |
| 196 | BB-22 6-HO-isoquinoline | isoquinolin-6-yl 1-(cyclohexylmethyl)-1H-indole-3- |
| | isomer | carboxylate |
| 197 | BB-22 6-HO-quinoline | quinolin-6-yl 1-(cyclohexylmethyl)-1H-indole-3- |
| | isomer | carboxylate |
| 198 | BB-22 7-HO-isoquinoline | isoquinolin-7-yl 1-(cyclohexylmethyl)-1H-indole-3- |
| | isomer | carboxylate |
| 199 | BB-22 7-HO-quinoline | quinolin-7-yl 1-(cyclohexylmethyl)-1H-indole-3- |
| | isomer | carboxylate |
| 200 | 5-fluoro BEPIRAPIM | (4-benzylpiperazin-1-yl)(1-(5-fluoropentyl)-1H-indol-3- |
| | (hydrochloride) | yl)methanone, monohydrochloride |
| 201 | 3-CAF | naphthalen-2-yl 1-(2-fluorophenyl)-1H-indazole-3- |
| | | carboxylate |
| 202 | CBL-018 | naphthalen-1-yl 1-pentyl-1H-indole-3-carboxylate |
| 203 | 4-cyano CUMYL- | 1-(4-cyanobutyl)-N-(1-methyl-1-phenylethyl)-1H- |
| | BUTINACA | indazole-3-carboxamide |
| 204 | 4-cyano CUMYL- | 2-(4-cyanobutyl)-N-(2-phenylpropan-2-yl)-2H-indazole- |
| | BUTINACA isomer 2 | 3-carboxamide |
| 205 | CUMYL-PICA | N-(1-methyl-1-phenylethyl)-1-pentyl-1H-indole-3- |
| | | carboxamide |
| 206 | 5-fluoro CUMYL-PICA | 1-(5-fluoropentyl)-N-(1-methyl-1-phenylethyl)-1H- |
| | | indole-3-carboxamide |
| 207 | CUMYL-THPINACA | N-(1-methyl-1-phenylethyl)-1-[(tetrahydro-2H-pyran-4- |
| | | yl)methyl]-1H-indazole-3-carboxamide |
| 208 | 5-fluoro CUMYL-P7AICA | 1-(5-fluoropentyl)-N-(2-phenylpropan-2-yl)-1H- |
| | | pyrrolo[2,3-b]pyridine-3-carboxamide |
| 209 | 5-fluoro CUMYL-PINACA | 1-(5-fluoropentyl)-N-(1-methyl-1-phenylethyl)-1H- |
| | | indazole-3-carboxamide |
| 210 | 5-fluoro CYPPICA | N-(cyclopropylmethyl)-1-(5-fluoropentyl)-1H-indole-3- |
| | | carboxamide |
| 211 | EAM2201 | (4-ethyl-1-naphthalenyl)[1-(5-fluoropentyl)-1H-indol-3- |
| | | yl]-methanone |
| 212 | EG 018 | 1-naphthalenyl(9-pentyl-9H-carbazol-3-yl)methanone |
| 213 | EG2201 | (9-(5-fluoropentyl)-9H-carbazol-3-yl)(naphthalen-1- |
| | | yl)methanone |
| 214 | F2201 | (4-fluoro-1-naphthalenyl)[1-(5-fluoropentyl)-1H-indol-3- |
| | | yl]-methanone |
| 215 | FAB-144 | (1-(5-fluoropentyl)-1H-indazol-3-yl)(2,2,3,3- |
| | | tetramethylcyclopropyl)methanone |
| 216 | FDU-NNEI | 1-(4-fluorobenzyl)-N-(naphthalen-1-yl)-1H-indole-3- |
| | | carboxamide |
| 217 | FDU-PB-22 | 1-[(4-fluorophenyl)methyl]-1H-indole-3-carboxylic acid, |
| | | 1-naphthalenyl ester |
| 218 | FUB-144 | (1-(4-fluorobenzyl)-1H-indol-3-yl)(2,2,3,3- |
| | | tetramethylcyclopropyl)methanone |
| 219 | FUB-NPB-22 | quinolin-8-yl 1-(4-fluorobenzyl)-1H-indazole-3- |
| | | carboxylate |
| 220 | FUB-PB-22 | 1-[(4-fluorophenyl)methyl]-1H-indole-3-carboxylic acid, |
| | | 8-quinolinyl ester |
| 221 | EMB-FUBINACA | ethyl (1-(4-fluorobenzyl)-1H-indazole-3-carbonyl)-L- |
| | | valinate |
| 222 | 5-Fluoropentyl-3- | (1-(5-fluoropentyl)-1H-indol-3-yl)(pyridin-3- |
| | pyridinoylindole | yl)methanone, monohydrochloride |
| 223 | IMMA | 2-[1-(4-chlorobenzoyl)-5-methoxy-2-methyl-1H-indol-3- |
| | | yl]-1-(4-morpholinyl)-ethanone |
| 224 | JWH 007 | (2-methyl-1-pentyl-1H-indol-3-yl)-1-naphthalenyl- |
| | | methanone |
| 225 | JWH 011 | [2-methyl-1-(1-methylhexyl)-1H-indol-3-yl]-1- |
| | | naphthalenyl-methanone |
| 226 | JWH 016 | (1-butyl-2-methyl-1H-indol-3-yl)-1-naphthalenyl- |
| | | methanone |
| 227 | JWH 018 | N-(4,5-epoxypentyl) analog naphthalen-1-yl(1-(3- |
| | | (oxiran-2-yl)propyl)-1H-indol-3-yl)methanone |
| 228 | JWH 018 | (1-pentyl-1H-indol-3-yl)-1-naphthalenyl-methanone |
| 229 | JWH 018 2′-naphthyl | 2-naphthalenyl(1-pentyl-1H-indol-3-yl)-methanone |
| | isomer | |
| 230 | JWH 018 2′-naphthyl-N- | (1-(3-methylbutan-2-yl)-1H-indol-3-yl)(naphthalen-2- |
| | (1,2-dimethylbutyl) isomer | yl)methanone |
| 231 | JWH 018 2′-naphthyl-N-(1- | [1-(1-ethylpropyl)-1H-indol-3-yl]-2-naphthalenyl- |
| | ethylpropyl) isomer | methanone |
| 232 | JWH 018 2′-naphthyl-N-(1- | naphthalen-2-yl(1-(pentan-2-yl)-1H-indol-3- |
| | methylbutyl) isomer | yl)methanone |
| 233 | JWH 018 2′-naphthyl-N- | [1-(2,2-dimethylpropyl)-1H-indol-3-yl]-2-naphthalenyl- |
| | (2,2-dimethylbutyl) isomer | methanone |
| 234 | JWH 018 2′-naphthyl-N-(2- | (1-(2-methylbutyl)-1H-indol-3-yl)(naphthalen-2-yl)- |
| | methylbutyl) isomer | methanone |
| 235 | JWH 018 2′-naphthyl-N-(3- | (1-isopentyl-1H-indol-3-yl)(naphthalen-2-yl)methanone |
| | methylbutyl) isomer | |
| 236 | JWH 018 6-CH₃O-indole | (6-methoxy-1-pentyl-1H-indol-3-yl)(naphthalen-1-yl)- |
| | analog | methanone |
| 237 | JWH 018 8-quinolinyl | 1-pentyl-N-8-quinolinyl-1H-indole-3-carboxamide |
| | carboxamide | |
| 238 | JWH 018 adamantyl analog | (1s,3s)-adamantan-1-yl(1-pentyl-1H-indol-3- |
| | | yl)methanone |
| 239 | JWH 018 adamantyl | 1-pentyl-N-tricyclo[3.3.1.1]dec-1-yl-1H-indole-3- |
| | carboxamide | carboxamide |
| 240 | JWH 018 benzimidazole | naphthalen-1-yl(1-pentyl-1H-benzo[d]imidazol-2- |
| | analog | yl)methanone |
| 241 | JWH 018 N-(1,1- | naphthalen-1-yl(1-(tert-pentyl)-1H-indol-3-yl)methanone |
| | dimethylpropyl) isomer | |
| 242 | JWH 018 N-(1,2- | (1-(3-methylbutan-2-yl)-1H-indol-3-yl)(naphthalen-1- |
| | dimethylpropyl) isomer | yl)methanone |
| 243 | JWH 018 N-(1-ethylpropyl) | naphthalen-1-yl(1-(pentan-3-yl)-1H-indol-3- |
| | isomer | yl)methanone |
| 244 | JWH 018 N-(1-methylbutyl) | naphthalen-1-yl(1-(pentan-2-yl)-1H-indol-3- |
| | isomer | yl)methanone |
| 245 | JWH 018 N-(2,2- | naphthalen-1-yl(1-neopentyl-1H-indol-3-yl)-methanone |
| | dimethylpropyl) isomer | |
| 246 | JWH 018 N-(2-methylbutyl) | N-(2-methylbutyl)-3-(1-naphthoyl)-indole |
| | isomer | |
| 247 | JWH 018 N-(3-methylbutyl) | N-(3-Methylbutyl)-3-(1-naphthoyl)-indole |
| | isomer | |
| 248 | JWH 018 N-(5- | (1-(5-bromopentyl)-1H-indol-3-yl)(naphthalen-1- |
| | bromopentyl) analog | yl)methanone |
| 249 | JWH 018 N-(5- | (1-(5-chloropentyl)-1H-indol-3-yl)(naphthalen-1- |
| | chloropentyl) analog | yl)methanone |
| 250 | 5-fluoro JWH 018 | [1-(5-fluoropentyl)-1H-indol-3-yl]tricyclo[3.3.1.13,7]dec- |
| | adamantyl analog | 1-yl-methanone |
| 251 | FUB-JWH 018 | (1-(4-fluorobenzyl)-1H-indol-3-yl)(naphthalen-1- |
| | | yl)methanone |
| 252 | JWH 019 | (1-hexyl-1H-indol-3-yl)-1-naphthalenyl-methanone |
| 253 | JWH 019 N-(2-fluorohexyl) | (1-(2-fluorohexyl)-1H-indol-3-yl)(naphthalen-1- |
| | isomer | yl)methanone |
| 254 | JWH 019 N-(3-fluorohexyl) | (1-(3-fluorohexyl)-1H-indol-3-yl)(naphthalen-1- |
| | isomer | yl) methanone |
| 255 | JWH 019 N-(4-fluorohexyl) | (1-(4-fluorohexyl)-1H-indol-3-yl)(naphthalen-1- |
| | isomer | yl)methanone |
| 256 | JWH 019 N-(5-fluorohexyl) | (1-(5-fluorohexyl)-1H-indol-3-yl)(naphthalen-1- |
| | isomer | yl)methanone |
| 257 | JWH 019 N-(6-fluorohexyl) | (1-(6-fluorohexyl)-1H-indol-3-yl)(naphthalen-1- |
| | isomer | yl)methanone |
| 258 | JWH 022 | 1-naphthalenyl[1-(4-penten-1-yl)-1H-indol-3-yl]- |
| | | methanone |
| 259 | JWH 030 | 1-naphthalenyl(1-pentyl-1H-pyrrol-3-yl)-methanone |
| 260 | JWH 030 2-naphthoyl | naphthalen-1-yl(1-pentyl-1H-pyrrol-2-yl)methanone |
| | isomer | |
| 261 | JWH 031 | (1-hexyl-1H-pyrrol-3-yl)-1-naphthalenyl-methanone |
| 262 | JWH 031 2′-isomer | (1-hexyl-1H-pyrrol-2-yl)-1-naphthalenyl-methanone |
| 263 | JWH 071 | (1-ethyl-1H-indol-3-yl)-1-naphthalenyl-methanone |
| 264 | JWH 072 | 1-naphthalenyl(1-propyl-1H-indol-3-yl)-methanone |
| 265 | JWH 073 | (1-butyl-1H-indol-3-yl)-1-naphthalenyl-methanone |
| 266 | JWH 073 2-CH₃-naphthyl | (1-butyl-1H-indol-3-yl)(2-methylnaphthalen-1- |
| | analog | yl)methanone |
| 267 | JWH 073 2′-naphthyl | (1-butyl-1H-indol-3-yl)(naphthalen-2-yl)-methanone |
| | isomer | |
| 268 | JWH 073 2′-naphthyl-N-(1- | (1-(sec-butyl)-1H-indol-3-yl)(naphthalen-2-yl)- |
| | methylpropyl) isomer | methanone |
| 269 | JWH 073 2′-naphthyl-N-(2- | (1-isobutyl-1H-indol-3-yl)(naphthalen-2-yl)methanone |
| | methylpropyl) isomer | |
| 270 | JWH 073 4-CH₃-naphthyl | (1-butyl-1H-indol-3-yl)(4-methylnaphthalen-1- |
| | analog | yl)methanone |
| 271 | JWH 073 6-CH₃O-indole | (1-butyl-6-methoxy-1H-indol-3-yl)-1-naphthalenyl- |
| | analog | methanone |
| 272 | JWH 073 N-(1,1- | (1-(tert-butyl)-1H-indol-3-yl)(naphthalen-1- |
| | dimethylethyl) isomer | yl)methanone |
| 273 | JWH 073 N-(1-CH₃-propyl) | (1-(sec-butyl)-1H-indol-3-yl)(naphthalen-1- |
| | isomer | yl) methanone |
| 274 | JWH 073 N-(2- | (1-isobutyl-1H-indol-3-yl)(naphthalen-1-yl)methanone |
| | methylpropyl) isomer | |
| 275 | JWH 080 | (1-butyl-1H-indol-3-yl)(4-methoxy-1-naphthalenyl)- |
| | | methanone |
| 276 | JWH 081 | (4-methoxy-1-naphthalenyl)(1-pentyl-1H-indol-3-yl)- |
| | | methanone |
| 277 | JWH 081 2-CH₃O-naphthyl | (2-methoxy-1-naphthalenyl)(1-pentyl-1H-indol-3-yl)- |
| | isomer | methanone |
| 278 | JWH 081 3-CH₃O-naphthyl | (3-methoxynaphthalen-1-yl)(1-pentyl-1H-indol-3- |
| | isomer | yl)methanone |
| 279 | JWH 081 5-CH₃O-naphthyl | (5-methoxynaphthalen-1-yl)(1-pentyl-1H-indol-3- |
| | isomer | yl)methanone |
| 280 | JWH 081 6-CH₃O-naphthyl | (6-methoxy-1-naphthalenyl)(1-pentyl-1H-indol-3-yl)- |
| | isomer | methanone |
| 281 | JWH 081 7-CH₃O-naphthyl | (7-methoxy-1-naphthalenyl)(1-pentyl-1H-indol-3-yl)- |
| | isomer | methanone |
| 282 | JWH 081 N- | [1-(cyclohexylmethyl)-1H-indol-3-yl](4-methoxy-1- |
| | (cyclohexylmethyl) analog | naphthalenyl)-methanone |
| 283 | JWH 098 | (4-methoxy-1-naphthalenyl)(2-methyl-1-pentyl-1H- |
| | | indol-3-yl)-methanone |
| 284 | JWH 116 | (2-ethyl-1-pentyl-1H-indol-3-yl)-1-naphthalenyl- |
| | | methanone |
| 285 | JWH 122 | (4-methyl-1-naphthalenyl)(1-pentyl-1H-indol-3-yl)- |
| | | methanone |
| 286 | JWH 122 2-CH₃-naphthyl | (2-methylnaphthalen-1-yl)(1-pentyl-1H-indol-3- |
| | isomer | yl)methanone |
| 287 | JWH 122 3-CH₃-naphthyl | (3-methylnaphthalen-1-yl)(1-pentyl-1H-indol-3- |
| | isomer | yl)methanone |
| 288 | JWH 122 5-CH₃-naphthyl | (5-methylnaphthalen-1-yl)(1-pentyl-1H-indol-3- |
| | isomer | yl)methanone |
| 289 | JWH 122 6-CH₃-naphthyl | (6-methylnaphthalen-1-yl)(1-pentyl-1H-indol-3- |
| | isomer | yl)methanone |
| 290 | JWH 122 7-CH₃-naphthyl | (7-methyl-1-naphthalenyl)(1-pentyl-1H-indol-3-yl)- |
| | isomer | methanone |
| 291 | JWH 122 8-CH₃-naphthyl | (8-methylnaphthalen-1-yl)(1-pentyl-1H-indol-3- |
| | isomer | yl)methanone |
| 292 | JWH 122 N-(4-pentenyl) | (4-methylnaphthalen-1-yl)(1-(pent-4-en-1-yl)-1H-indol- |
| | analog | 3-yl)methanone |
| 293 | JWH 145 | 1-naphthalenyl(1-pentyl-5-phenyl-1H-pyrrol-3-yl)- |
| | | methanone |
| 294 | JWH 146 | (1-heptyl-5-phenyl-1H-pyrrol-3-yl)-1-naphthalenyl- |
| | | methanone |
| 295 | JWH 147 | (1-hexyl-5-phenyl-1H-pyrrol-3-yl)-1-naphthalenyl- |
| | | methanone |
| 296 | JWH 149 | (4-methyl-1-naphthalenyl)(2-methyl-1-pentyl-1H-indol- |
| | | 3-yl)-methanone |
| 297 | JWH 167 | 1-(1-pentyl-1H-indol-3-yl)-2-phenyl-ethanone |
| 298 | JWH 175 | 3-(1-naphthalenylmethyl)-1-pentyl-1H-indole |
| 299 | JWH 180 | (1-propyl-1H-indol-3-yl)(4-propyl-1-naphthalenyl)- |
| | | methanone |
| 300 | JWH 182 | (1-pentyl-1H-indol-3-yl)(4-propyl-1-naphthalenyl)- |
| | | methanone |
| 301 | JWH 193 | (4-methyl-1-naphthalenyl)[1-[2-(4-morpholinyl)ethyl]- |
| | | 1H-indol-3-yl]-methanone |
| 302 | JWH 198 | (4-methoxy-1-naphthalenyl)[1-[2-(4-morpholinyl)ethyl]- |
| | | 1H-indol-3-yl]-methanone |
| 303 | JWH 200 | [1-[2-(4-morpholinyl)ethyl]-1H-indol-3-yl]-1- |
| | | naphthalenyl-methanone |
| 304 | JWH 200 2′-naphthyl | [1-[2-(4-morpholinyl)ethyl]-1H-indol-3-yl]-2- |
| | isomer | naphthalenyl-methanone |
| 305 | JWH 200 analog 1 | (E)-3,4,4-trimethyl-1-(1-(2-morpholinoethyl)-1H-indol-3- |
| | | yl)pent-2-en-1-one |
| 306 | JWH 201 | 2-(4-methoxyphenyl)-1-(1-pentyl-1H-indol-3-yl)- |
| | | ethanone |
| 307 | JWH 203 | 2-(2-chlorophenyl)-1-(1-pentyl-1H-indol-3-yl)-ethanone |
| 308 | JWH 203 3-Cl-phenyl | 2-(3-chlorophenyl)-1-(1-pentyl-1H-indol-3-yl)-ethanone |
| | isomer | |
| 309 | JWH 203 4-Cl-phenyl | 2-(4-chlorophenyl)-1-(1-pentyl-1H-indol-3-yl)-ethanone |
| | isomer | |
| 310 | JWH 210 | (4-ethyl-1-naphthalenyl)(1-pentyl-1H-indol-3-yl)- |
| | | methanone |
| 311 | JWH 210 2-Et-naphthyl | (2-ethylnaphthalen-1-yl)(1-pentyl-1H-indol-3- |
| | isomer | yl)methanone |
| 312 | JWH 210 3-Et-naphthyl | (3-ethyl-1-naphthalenyl)(1-pentyl-1H-indol-3-yl)- |
| | isomer | methanone |
| 313 | JWH 210 5-Et-naphthyl | (5-ethyl-1-naphthalenyl)(1-pentyl-1H-indol-3-yl)- |
| | isomer | methanone |
| 314 | JWH 210 6-Et-naphthyl | (6-ethylnaphthalen-1-yl)(1-pentyl-1H-indol-3- |
| | isomer | yl)methanone |
| 315 | JWH 210 7-Et-naphthyl | (7-ethyl-1-naphthalenyl)(1-pentyl-1H-indol-3-yl)- |
| | isomer | methanone |
| 316 | JWH 210 8-Et-naphthyl | (8-ethylnaphthalen-1-yl)(1-pentyl-1H-indol-3- |
| | isomer | yl)methanone |
| 317 | JWH 213 | (4-ethyl-1-naphthalenyl)(2-methyl-1-pentyl-1H-indol-3- |
| | | yl)-methanone |
| 318 | JWH 249 | 2-(2-bromophenyl)-1-(1-pentyl-1H-indol-3-yl)-ethanone |
| 319 | JWH 250 | 1-(1-pentyl-1H-indol-3-yl)-2-(2-methoxyphenyl)- |
| | | ethanone |
| 320 | JWH 251 | 2-(2-methylphenyl)-1-(1-pentyl-1H-indol-3-yl)-ethanone |
| 321 | JWH 251 3-CH₃-phenyl | 1-(1-pentyl-1H-indol-3-yl)-2-(m-tolyl)ethanone |
| | isomer | |
| 322 | JWH 251 4-CH₃-phenyl | 2-(4-methylphenyl)-1-(1-pentyl-1H-indol-3-yl)-ethanone |
| | isomer | |
| 323 | JWH 302 | 2-(3-methoxyphenyl)-1-(1-pentyl-1H-indol-3-yl)- |
| | | ethanone |
| 324 | JWH 307 | [5-(2-fluorophenyl)-1-pentyl-1H-pyrrol-3-yl]-1- |
| | | naphthalenyl-methanone |
| 325 | JWH 309 | 1-naphthalenyl[5-(1-naphthalenyl)-1-pentyl-1H-pyrrol-3- |
| | | yl]-methanone |
| 326 | JWH 368 | [5-(3-fluorophenyl)-1-pentyl-1H-pyrrol-3-yl]-1- |
| | | naphthalenyl-methanone |
| 327 | JWH 369 | [5-(2-chlorophenyl)-1-pentyl-1H-pyrrol-3-yl]-1- |
| | | naphthalenyl-methanone |
| 328 | JWH 370 | [5-(2-methylphenyl)-1-pentyl-1H-pyrrol-3-yl]-1- |
| | | naphthalenyl-methanone |
| 329 | JWH 387 | (4-bromo-1-naphthalenyl)(1-pentyl-1H-indol-3-yl)- |
| | | methanone |
| 330 | JWH 398 | (4-chloro-1-naphthalenyl)(1-pentyl-1H-indol-3-yl)- |
| | | methanone |
| 331 | JWH 398 2-Cl-naphthyl | (2-chloronaphthalen-1-yl)(1-pentyl-1H-indol-3- |
| | isomer | yl)methanone |
| 332 | JWH 398 3-Cl-naphthyl | (3-chloronaphthalen-1-yl)(1-pentyl-1H-indol-3- |
| | isomer (hydrate) | yl)methanone, hydrate |
| 333 | JWH 398 5-Cl-naphthyl | (5-chloronaphthalen-1-yl)(1-pentyl-1H-indol-3- |
| | isomer | yl)methanone |
| 334 | JWH 398 6-Cl-naphthyl | (6-chloronaphthalen-1-yl)(1-pentyl-1H-indol-3- |
| | isomer | yl)methanone |
| 335 | JWH 398 7-Cl-naphthyl | (7-chloronaphthalen-1-yl)(1-pentyl-1H-indol-3- |
| | isomer | yl)methanone |
| 336 | JWH 398 8-Cl-naphthyl | (8-chloronaphthalen-1-yl)(1-pentyl-1H-indol-3- |
| | isomer | yl)methanone |
| 337 | JWH 412 | (4-fluoro-1-naphthalenyl)(1-pentyl-1H-indol-3-yl)- |
| | | methanone |
| 338 | JWH 424 | (8-bromonaphthalen-1-yl)(1-pentyl-1H-indol-3- |
| | | yl)methanone |
| 339 | M-144 | (1-(5-fluoropentyl)-2-methyl-1H-indol-3-yl)(2,2,3,3- |
| | | tetramethylcyclopropyl)methanone |
| 340 | MAB-CHMINACA | N-[1-(aminocarbonyl)-2,2-dimethylpropyl]-1- |
| | | (cyclohexylmethyl)-1H-indazole-3-carboxamide |
| 341 | MA-CHMINACA | N-[[1-(cyclohexylmethyl)-1H-indazol-3-yl]carbonyl]-L- |
| | | valine, methyl ester |
| 342 | MAM2201 | [1-(5-fluoropentyl)-1H-indol-3-yl](4-methyl-1- |
| | | naphthalenyl)-methanone |
| 343 | MAM2201 N-(2- | (1-(2-fluoropentyl)-1H-indol-3-yl)(4-methylnaphthalen- |
| | fluoropentyl) isomer | 1-yl)methanone |
| 344 | MAM2201 N-(3- | (1-(3-fluoropentyl)-1H-indol-3-yl)(4-methylnaphthalen- |
| | fluoropentyl) isomer | 1-yl)methanone |
| 345 | MAM2201 N-(4- | (1-(4-fluoropentyl)-1H-indol-3-yl)(4-methylnaphthalen- |
| | fluoropentyl) isomer | 1-yl)methanone |
| 346 | MAM2201 N-(5- | (1-(5-chloropentyl)-1H-indol-3-yl)(4-methylnaphthalen- |
| | chloropentyl) analog | 1-yl)methanone |
| 347 | MCHB-1 | 1-(cyclohexylmethyl)-2-[(4-ethoxyphenyl)methyl]-N,N- |
| | | diethyl-1H-benzimidazole-5-carboxamide |
| 348 | MDA 19 | (2Z)-2-(1-hexyl-1,2-dihydro-2-oxo-3H-indol-3- |
| | | ylidene)hydrazide, benzoic acid |
| 349 | MDA 77 | 2Z-(1,2-dihydro-6-methoxy-2-oxo-1-pentyl-3H-indol-3- |
| | | ylidene)hydrazide benzoic acid |
| 350 | MDMB-CHMCZCA | methyl (S)-2-(9-(cyclohexylmethyl)-9H-carbazole-3- |
| | | carboxamido)-3,3-dimethylbutanoate |
| 351 | MDMB-CHMICA | N-[[1-(cyclohexylmethyl)-1H-indol-3-yl]carbonyl]-3- |
| | | methyl-L-valine, methyl ester |
| 352 | MDMB-CHMINACA | N-[[1-(cyclohexylmethyl)-1H-indazol-3-yl]carbonyl]-3- |
| | | methyl-L-valine, methyl ester |
| 353 | MDMB-FUBICA | N-[[1-[(4-fluorophenyl)methyl]-1H-indol-3-yl]carbonyl]-3- |
| | | methyl-L-valine, methyl ester |
| 354 | MDMB-FUBINACA | N-[[1-[(4-fluorophenyl)methyl]-1H-indazol-3- |
| | | yl]carbonyl]-3-methyl-L-valine, methyl ester |
| 355 | Mepirapim (hydrochloride) | (4-methylpiperazin-1-yl)(1-pentyl-1H-indol-3- |
| | | yl)methanone, monohydrochloride |
| 356 | MMB018 | N-[(1-pentyl-1H-indol-3-yl)carbonyl]-L-valine, methyl |
| | | ester |
| 357 | MMB2201 | N-[[1-(5-fluoropentyl)-1H-indol-3-yl]carbonyl]-L-valine, |
| | | methyl ester |
| 358 | MMB-CHMICA | methyl (1-(cyclohexylmethyl)-1H-indole-3-carbonyl)-L- |
| | | valinate |
| 359 | MMB-FUBICA | N-[[1-[(4-fluorophenyl)methyl]-1H-indol-3-yl]carbonyl]-L- |
| | | valine, methyl ester |
| 360 | MMB-FUBINACA | N-[[1-[(4-fluorophenyl)methyl]-1H-indazol-3- |
| | | yl]carbonyl]-L-valine, methyl ester |
| 361 | MN-18 | N-1-naphthalenyl-1-pentyl-1H-indazole-3-carboxamide |
| 362 | 5-fluoro MN-18 | 1-(5-fluoropentyl)-N-1-naphthalenyl-1H-indazole-3- |
| | | carboxamide |
| 363 | MN-25 | 7-methoxy-1-[2-(4-morpholinyl)ethyl]-N-[(1S,2S,4R)- |
| | | 1,3,3-trimethylbicyclo[2.2.1]hept-2-yl]-1H-indole-3- |
| | | carboxamide |
| 364 | MN-25-2-methyl derivative | 7-methoxy-2-methyl-1-[2-(4-morpholinyl)ethyl]-N- |
| | | [(1S,2S,4R)-1,3,3-trimethylbicyclo[2.2.1]hept-2-yl]-1H- |
| | | indole-3-carboxamide |
| 365 | MO-CHMINACA | 1-methoxy-3,3-dimethyl-1-oxobutan-2-yl 1- |
| | | (cyclohexylmethyl)-1H-indazole-3-carboxylate |
| 366 | NM2201 | naphthalen-1-yl 1-(5-fluoropentyl)-1H-indole-3- |
| | | carboxylate |
| 367 | 2-fluoro NNEI | 1-(2-fluoropentyl)-N-(naphthalen-1-yl)-1H-indole-3- |
| | | carboxamide |
| 368 | 3-fluoro NNEI | 1-(3-fluoropentyl)-N-(naphthalen-1-yl)-1H-indole-3- |
| | | carboxamide |
| 369 | 4-fluoro NNEI | 1-(4-fluoropentyl)-N-(naphthalen-1-yl)-1H-indole-3- |
| | | carboxamide |
| 370 | 5-fluoro NNEI | 1-(5-fluoropentyl)-N-(naphthalen-1-yl)-1H-indole-3- |
| | | carboxamide |
| 371 | 5-fluoro NNEI 2′-naphthyl | 1-(5-fluoropentyl)-N-(naphthalen-2-yl)-1H-indole-3- |
| | isomer | carboxamide |
| 372 | NNEI 1′-naphthyl isomer | N-1-naphthalenyl-1-pentyl-1H-indole-3-carboxamide |
| 373 | NNEI 2′-naphthyl isomer | N-2-naphthalenyl-1-pentyl-1H-indole-3-carboxamide |
| 374 | NNEI 2′-indazole isomer | N-(naphthalen-1-yl)-2-pentyl-2H-indazole-3- |
| | | carboxamide |
| 375 | NPB-22 | 1-pentyl-1H-indazole-3-carboxylic acid, 8-quinolinyl |
| | | ester |
| 376 | (+/−)-ORG 28611 | [1-(cyclohexylmethyl)-7-methoxy-1H-indol-3-yl](3,4- |
| | | dimethyl-1-piperazinyl)-methanone |
| 377 | 5-fluoro NPB-22 | 1-(5-fluoropentyl)-8-quinolinyl ester-1H-indazole-3- |
| | | carboxylic acid |
| 378 | 5-fluoro PB-22 | 7-hydroxyquinoline isomer quinolin-7-yl 1-(5- |
| | | fluoropentyl)-1H-indole-3-carboxylate |
| 379 | 5-fluoro PB-22 3-HO- | quinolin-3-yl 1-(5-fluoropentyl)-1H-indole-3-carboxylate |
| | quinoline isomer | |
| 380 | 5-fluoro PB-22 4-HO- | isoquinolin-4-yl 1-(5-fluoropentyl)-1H-indole-3- |
| | isoquinoline isomer | carboxylate |
| 381 | 5-fluoro PB-22 4-HO- | quinolin-4-yl 1-(5-fluoropentyl)-1H-indole-3-carboxylate |
| | quinoline isomer | |
| 382 | 5-fluoro PB-22 5-HO- | isoquinolin-5-yl 1-(5-fluoropentyl)-1H-indole-3- |
| | isoquinoline isomer | carboxylate |
| 383 | 5-fluoro PB-22 5-HO- | quinolin-5-yl 1-(5-fluoropentyl)-1H-indole-3-carboxylate |
| | quinoline isomer | |
| 384 | 5-fluoro PB-22 6-HO- | isoquinolin-6-yl 1-(5-fluoropentyl)-1H-indole-3- |
| | isoquinoline isomer | carboxylate |
| 385 | 5-fluoro PB-22 6-HO- | quinolin-6-yl 1-(5-fluoropentyl)-1H-indole-3-carboxylate |
| | quinoline isomer | |
| 386 | 5-fluoro PB-22 7-HO- | isoquinolin-7-yl 1-(5-fluoropentyl)-1H-indole-3- |
| | isoquinoline isomer | carboxylate |
| 387 | 5-fluoro PB-22 8-HO- | isoquinolin-8-yl 1-(5-fluoropentyl)-1H-indole-3- |
| | isoquinoline isomer | carboxylate |
| 388 | 5-fluoro PB-22 N-(2-F- | quinolin-8-yl 1-(2-fluoropentyl)-1H-indole-3-carboxylate |
| | pentyl) isomer | |
| 389 | 5-fluoro PB-22 N-(3- | quinolin-8-yl 1-(3-fluoropentyl)-1H-indole-3-carboxylate |
| | fluoropentyl) isomer | |
| 390 | 5-fluoro PB-22 N-(4-F- | quinolin-8-yl 1-(4-fluoropentyl)-1H-indole-3-carboxylate |
| | pentyl) isomer | |
| 391 | PB-22 4-HO-isoquinoline | isoquinolin-4-yl 1-pentyl-1H-indole-3-carboxylate |
| | isomer | |
| 392 | PB-22 4-HO-quinoline | quinolin-4-yl 1-pentyl-1H-indole-3-carboxylate |
| | isomer | |
| 393 | PB-22 5-HO-isoquinoline | isoquinolin-5-yl 1-pentyl-1H-indole-3-carboxylate |
| | isomer | |
| 394 | PB-22 5-HO-quinoline | quinolin-5-yl 1-pentyl-1H-indole-3-carboxylate |
| | isomer | |
| 395 | PB-22 6-HO-isoquinoline | isoquinolin-6-yl 1-pentyl-1H-indole-3-carboxylate |
| | isomer | |
| 396 | PB-22 6-HO-quinoline | quinolin-6-yl 1-pentyl-1H-indole-3-carboxylate |
| | isomer | |
| 397 | PB-22 7-HO-isoquinoline | isoquinolin-7-yl 1-pentyl-1H-indole-3-carboxylate |
| | isomer | |
| 398 | PB-22 7-HO-quinoline | quinolin-7-yl 1-pentyl-1H-indole-3-carboxylate |
| | isomer | |
| 399 | PB-22 8-HO-isoquinoline | isoquinolin-8-yl 1-pentyl-1H-indole-3-carboxylate |
| | isomer | |
| 400 | 5-fluoro PCN | 1-(5-fluoropentyl)-N-(naphthalen-1-yl)-1H-pyrrolo[3,2- |
| | | c]pyridine-3-carboxamide |
| 401 | PF-03550096 | N-[(1S)-1-(aminocarbonyl)-2,2-dimethylpropyl]-2,3- |
| | | dihydro-3-(3-hydroxy-3-methylbutyl)-2-oxo-1H- |
| | | benzimidazole-1-carboxamide |
| 402 | Pravadoline | (4-methoxyphenyl)[2-methyl]-1-[2-(4-morpholinyl)ethyl]- |
| | | 1H-indol-3-yl]-methanone |
| 403 | PTI-1 (hydrochloride) | N,N-diethyl-2-(1-pentyl-1H-indol-3-yl-4- |
| | | thiazolemethanamine |
| 404 | PTI-2 (hydrochloride) | N-(2-methoxyethyl)-N-(1-methylethyl)-2-(1-pentyl-1H- |
| | | indol-3-yl)-4-thiazolemethanamine, monohydrochloride |
| 405 | PX 1 | (S)-N-(1-amino-1-oxo-3-phenylpropan-2-yl)-1-(5- |
| | | fluoropentyl)-1H-indole-3-carboxamide |
| 406 | PX 2 | (S)-N-(1-amino-1-oxo-3-phenylpropan-2-yl)-1-(5- |
| | | fluoropentyl)-1H-indazole-3-carboxamide |
| 407 | 5-fluoro PY-PICA | (1-(5-fluoropentyl)-1H-indol-3-yl)(pyrrolidin-1- |
| | | yl)methanone |
| 408 | 5-fluoro PY-PINACA | (1-(5-fluoropentyl)-1H-indazol-3-yl)(pyrrolidin-1- |
| | | yl) methanone |
| 409 | RCS-4 | (4-methoxyphenyl)(1-pentyl-1H-indol-3-yl)methanone |
| 410 | RCS-4 2-methoxy isomer | (2-methoxyphenyl)(1-pentyl-1H-indol-3-yl)-methanone |
| 411 | RCS-4 3-methoxy isomer | (3-methoxyphenyl)(1-pentyl-1H-indol-3-yl)methanone |
| 412 | RCS-4-C4 homolog | (4-methoxyphenyl)(1-butyl-1H-indol-3-yl)-methanone |
| 413 | RCS-8 | 1-(1-(2-cyclohexylethyl)-1H-indol-3-yl)-2-(2- |
| | | methoxyphenyl)ethanone |
| 414 | RCS-8 4-methoxy isomer | 1-(1-(2-cyclohexylethyl)-1H-indol-3-yl)-2-(4- |
| | | methoxyphenyl)ethanone |
| 415 | RCS-8 3-methoxy isomer | 1-(1-(2-cyclohexylethyl)-1H-indol-3-yl)-2-(3- |
| | | methoxyphenyl)ethanone |
| 416 | SDB-005 | naphthalen-1-yl 1-pentyl-1H-indazole-3-carboxylate |
| 417 | 5-fluoro SDB-005 | naphthalen-1-yl 1-(5-fluoropentyl)-1H-indazole-3- |
| | | carboxylate |
| 418 | SDB-006 | 1-pentyl-N-(phenylmethyl)-1H-indole-3-carboxamide |
| 419 | 5-fluoro SDB-006 | 1-(5-fluoropentyl)-N-(phenylmethyl)-1H-indole-3- |
| | | carboxamide |
| 420 | SDB-006 N-phenyl analog | 1-pentyl-N-phenyl-1H-indole-3-carboxamide |
| 421 | SER-601 | 1,4-dihydro-6-(1-methylethyl)-4-oxo-1-pentyl-N- |
| | | tricyclo[3.3.1.1^{3,7}]dec-1-yl-3-quinolinecarboxamide |
| 422 | STS-135 | 1-(5-fluoropentyl)-N-tricyclo[3.3.1.13,7]dec-1-yl-1H- |
| | | indole-3-carboxamide |
| 423 | STS-135 | 1-(5-fluoropentyl)-N-tricyclo[3.3.1.13,7]dec-1-yl-1H- |
| | | indole-3-carboxamide |
| 424 | THJ 018 | 1-naphthalenyl(1-pentyl-1H-indazol-3-yl)-methanone |
| 425 | 5-bromo THJ 018 | (1-(5-bromopentyl)-1H-indazol-3-yl)(naphthalen-1- |
| | | yl) methanone |
| 426 | 5-chloro THJ 018 | (1-(5-chloropentyl)-1H-indazol-3-yl)(naphthalen-1- |
| | | yl)methanone |
| 427 | THJ2201 | [1-(5-fluoropentyl)-1H-indazol-3-yl]-1-naphthalenyl- |
| | | methanone |
| 428 | THJ2201 (quinolinylamine | 1-(5-fluoropentyl)-N-(quinolin-8-yl)-1H-indazole-3- |
| | analog) | carboxamide |
| 429 | THJ2201 (desfluoro, | 1-pentyl-N-(quinolin-8-yl)-1H-indazole-3-carboxamide |
| | quinolinylamine analog | |
| 430 | UR-144 | (1-pentyl-1H-indol-3-yl)(2,2,3,3-tetramethylcyclopropyl)- |
| | | methanone |
| 431 | UR-144 N-(2-chloropentyl) | (1-(2-chloropentyl)-1H-indol-3-yl)(2,2,3,3- |
| | analog | tetramethylcyclopropyl)methanone |
| 432 | UR-144 N-(3-chloropentyl) | (1-(3-chloropentyl)-1H-indol-3-yl)(2,2,3,3- |
| | analog | tetramethylcyclopropyl)methanone |
| 433 | UR-144 N-(4-Cl-pentyl) | (1-(4-chloropentyl)-1H-indol-3-yl)(2,2,3,3- |
| | analog | tetramethylcyclopropyl)methanone |
| 434 | UR-144 N-(5-bromopentyl) | [1-(5-bromopentyl)-1H-indol-3-yl](2,2,3,3- |
| | analog | tetramethylcyclopropyl)-methanone |
| 435 | UR-144 N-(5-chloropentyl) | (1-(5-chloropentyl)-1H-indol-3-yl)(2,2,3,3- |
| | analog | tetramethylcyclopropyl)methanone |
| 436 | UR-144 N-(5-methylhexyl) | (1-(5-methylhexyl)-1H-indol-3-yl)(2,2,3,3- |
| | analog | tetramethylcyclopropyl)methanone |
| 437 | UR-144 N-heptyl analog | (1-heptyl-1H-indol-3-yl)(2,2,3,3-tetramethylcyclopropyl)- |
| | | methanone |
| 438 | URB447 | [4-amino-1-[(4-chlorophenyl)methyl]-2-methyl-5-phenyl- |
| | | 1H-pyrrol-3-yl]phenyl-methanone |
| 439 | WIN 54,461 | [6-bromo-2-methyl-1-[2-(4-morpholinyl)ethyl]-1H-indol- |
| | | 3-yl](4-methoxyphenyl)-methanone |
| 440 | (+)-WIN 55,212-2 | [(3R)-2,3-dihydro-5-methyl-3-(4- |
| | (mesylate) | morpholinylmethyl)pyrrolo[1,2,3-de]-1,4-benzoxazin-6- |
| | | yl]-1-naphthalenyl-methanone, methanesulfonate |
| 441 | (+/−)-WIN 55,212 | [2,3-dihydro-5-methyl-3-(4- |
| | (mesylate) | morpholinylmethyl)pyrrolo[1,2,3-de]-1,4-benzoxazin-6- |
| | | yl]-1-naphthalenyl-methanone, methanesulfonate |
| 442 | XLR11 | (1-(5-fluoropentyl)-1H-indol-3-yl)(2,2,3,3- |
| | | tetramethylcyclopropyl)methanone |
| 443 | XLR11 N-(2-fluoropentyl) | [1-(2-fluoropentyl)-1H-indol-3-yl](2,2,3,3- |
| | isomer | tetramethylcyclopropyl)-methanone |
| 444 | XLR11 N-(3-fluoropentyl) | [1-(3-fluoropentyl)-1H-indol-3-yl](2,2,3,3- |
| | isomer | tetramethylcyclopropyl)-methanone |
| 445 | XLR11 N-(4-fluuoropentyl) | (1-(4-fluoropentyl)-1H-indol-3-yl)(2,2,3,3- |
| | isomer | tetramethylcyclopropyl)methanone |
| 446 | XLR11 N-(4-pentenyl) | (1-(pent-4-en-1-yl)-1H-indol-3-yl)(2,2,3,3- |
| | analog | tetramethylcyclopropyl)methanone |
| 447 | XLR12 | (2,2,3,3-tetramethylcyclopropyl)[1-(4,4,4-trifluorobutyl)- |
| | | 1H-indol-3-yl]-methanone |

in one aspect, the invention provides a method wherein the cannabinoid derivative is a compound of Formula I, or a pharmaceutically acceptable salt thereof;

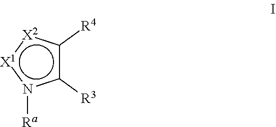

Wherein X¹ is —CR⁵—, nitrogen or —NR⁵—;
Wherein X² is —CR²—, nitrogen or —NR⁵—; provided X² is not nitrogen or —NR⁵— if X¹ is nitrogen or —NR⁵—;
Wherein R^{a} is absent, H, or R¹;
Wherein R¹ is selected from alkyl, haloalkyl, cyanoalkyl, alkenyl, heterocyclyl, cycloalkylalkyl, heterocyclylalkyl, arylalkyl and aryl;
Wherein R² is selected from H, aryl, aminocarbonylalkylaminocarbonyl, alkoxycarbonylalkylaminocarbonyl, aminocarbonyl(arylalkyl)aminocarbonyl, alkenylcarbonyl, arylcarbonyl, cycloalkylcarbonyl, arylalkylcarbonyl, heterocyclylalkylcarbonyl, heterocyclylcarbonyl, alkoxycarbonylalkyloxycarbonyl, cycloalkyloxycarbonyl, aryloxycarbonyl, heterocyclyloxycarbonyl, arylaminocarbonyl, arylalkylaminocarbonyl, heterocyclylaminocarbonyl, cycloalkylaminocarbonyl, optionally substituted arylalkyl and heterocyclylcarbonylalkyl;
Wherein R³ is H or aryl;
Wherein R⁴ is H or amino;
Wherein R⁴ and R³ together form a 6-membered aryl or heteroaryl ring, wherein the ring is optionally substituted with one or more substituents selected from halo, nitro, C₁₋₆-alkoxy, alkylaminocarbonyl, or optionally substituted C₆₋₁₀ aryl; and
Wherein R⁵ is selected from H, alkyl, arylcarbonyl, aminocarbonylalkylaminocarbonyl, arylalkyl, haloalkyl, cycloalkylalkyl, and cyanoalkyl; or a pharmaceutically acceptable salt thereof.

In one aspect, the invention provides a method wherein the cannabinoid derivative is a compound of Formula II

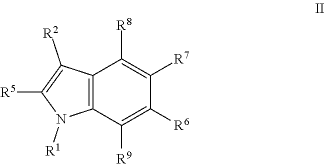

Wherein R¹ is selected from alkyl, haloalkyl, cyanoalkyl, alkenyl, cycloalkylalkyl, heterocyclyl, heterocyclylalkyl, optionally substituted arylalkyl and optionally substituted aryl; R² is aminocarbonylalkylaminocarbonyl, alkoxycarbonylalkylaminocarbonyl, aminocarbonyl(optionally substituted arylalkyl)aminocarbonyl, alkenylcarbonyl, optionally substituted arylcarbonyl, optionally substituted arylalkylcarbonyl, heterocyclylalkylcarbonyl, heterocyclylcarbonyl, heterocyclyloxycarbonyl, optionally substituted arylaminocarbonyl, heterocyclylaminocarbonyl, cycloalkylaminocarbonyl, cycloalkylcarbonyl, optionally substituted arylalkyl, and heterocyclylcarbonylalkyl; R⁵ is H, alkyl or arylcarbonyl; R⁶ is H, halo, nitro, C₁₋₆-alkoxy or optionally substituted C₆₋₁₀ aryl; R⁷ is H; R⁸ is H; and R⁹ is H; or pharmaceutically acceptable salt thereof.

In some embodiments, the method comprises compounds of Formula II wherein R¹ is selected from C₁₋₈-alkyl, C₁₋₆-haloalkyl, C₁₋₆-cyanoalkyl, C₂₋₆-alkenyl, C₃₋₈-cycloalkyl-C₁₋₆-alkyl, 3-8 membered heterocyclyl, 3-8 membered heterocyclyl-C₁₋₆ alkyl, optionally substituted phenyl-C₁₋₆-alkyl and optionally substituted phenyl; R² is aminocarbonyl(C₁₋₆-alkyDaminocarbonyl, C₁₋₆-alkoxycarbonyl(C₁₋₆-alkyl)aminocarbonyl, aminocarbonyl(optionally substituted phenyl-C₁₋₆-alkyl)aminocarbonyl, C₂₋₈-alkenylcarbonyl, optionally substituted C₆₋₁₀-arylcarbonyl, optionally substituted phenyl-C₁₋₆-alkylcarbonyl, 3-10 membered heterocyclyl-C₁₋₆-alkylcarbonyl, 3-10 membered heterocyclylcarbonyl, 3-10 membered heterocyclyloxycarbonyl, optionally substituted C₆₋₁₀-arylaminocarbonyl, 3-10 membered heterocyclylaminocarbonyl, Cm-cycloalkylaminocarbonyl, Cm-cycloalkylcarbonyl, optionally substituted C₆₋₁₀-aryl-C₁₋₆-alkyl, and 3-10 membered heterocyclylcarbonyl-C₁₋₆-alkyl; R⁵ is H, C₁₋₆-alkyl or arylcarbonyl; and R⁶ is H, halo, nitro, C₁₋₆-alkoxy or optionally substituted C₆₋₁₀ aryl; or pharmaceutically acceptable salt thereof.

In some embodiments, the method comprises compounds of Formula II wherein R¹ is selected from propyl, butyl, pentyl, 1-methylpropyl, 2-methylpropyl, 1,1-dimethylpropyl, 1,2-dimethylpropyl, 2,2-dimethylpropyl, tert-butyl, 1-methylbutyl, 2-methylbutyl, 3-methylbutyl, 1-ethylpropyl, hexyl, 1-methylhexyl, 5-methylhexyl, heptyl, 3-chlorobutyl, 2-chloropentyl, 3-chloropentyl, 4-chloropentyl, 5-chloropentyl, 2-fluoropentyl, 3-fluoropentyl, 4-fluoropentyl, 5-fluoropentyl, 5-bromopentyl, 2-chlorohexyl, 3-chlorohexyl, 4-chlorohexyl, 5-chlorohexyl, 6-chlorohexyl, 4-cyanobutyl, penten-4-yl, cyclohexylmethyl, cyclohexylethyl, 1-methyl-azepan-3-yl, 4-morpholinylmethyl, 4-morpholinylethyl, 2-oxiranylpropyl, 1-methylpiperidin-2-ylmethyl, 4-fluorophenylmethyl and 4-chlorophenylcarbonyl; R² is aminocarbonyl-(2,2-dimethylpropyl)aminocarbonyl, aminocarbonyl-(2-methylpropyl)aminocarbonyl, methoxycarbonyl-(2,2-dimethylethyl)aminocarbonyl, methoxycarbonyl-(2,2-dimethylpropyl)aminocarbonyl, methoxycarbonyl-(2-methylpropyl)aminocarbonyl, aminocarbonyl(phenylethyl)aminocarbonyl, 2,3,3-trimethyl-1-but-1-enylcarbonyl, 4-hydroxyphenylcarbonyl, 2-iodo-5-nitophenylcarbonyl, 2-iodophenylcarbonyl, 3-iodophenylcarbonyl, 4-iodophenylcarbonyl, 2-methoxyphenylcarbonyl, 3-methoxyphenylcarbonyl, 4-methoxyphenylcarbonyl, 1-naphthylcarbonyl, 2-naphthylcarbonyl, 1-methoxy-4-naphthylcarbonyl, 2-methoxy-1-naphthylcarbonyl, 3-methoxy-1-naphthylcarbonyl, 5-methoxy-1-naphthylcarbonyl, 6-methoxy-1-naphthylcarbonyl, 7-methoxy-1-naphthylcarbonyl, 1-methyl-4-naphthylcarbonyl, 2-methyl-1-naphthylcarbonyl, 3-methyl-1-naphthylcarbonyl, 4-methyl-1-naphthylcarbonyl, 5-methyl-1-naphthylcarbonyl, 6-methyl-1-naphthylcarbonyl, 7-methyl-1-naphthylcarbonyl, 8-methyl-1-naphthylcarbonyl, 2-ethyl-1-naphthylcarbonyl, 3-ethyl-1-naphthylcarbonyl, 5-ethyl-1-naphthylcarbonyl, 6-ethyl-1-naphthylcarbonyl, 7-ethyl-1-naphthylcarbonyl, 8-ethyl-1-naphthylcarbonyl, 1-ethyl-4-naphthylcarbonyl, 1-propyl-4-naphthylcarbonyl, 2-propyl-1-naphthylcarbonyl, 1-fluoro-4-naphthylcarbonyl, 1-bromo-4-naphthylcarbonyl, 8-bromo-1-naphthylcarbonyl, 1-chloro-4-naphthylcarbonyl, 2-chloro-1-naphthylcarbonyl, 3-chloro-1-naphthylcarbonyl, 5-chloro-1-naphthylcarbonyl, 6-chloro-1-naphthylcarbonyl, 7-chloro-1-naphthylcarbonyl, 8-chloro-1-naphthylcarbonyl, 4-hydroxyethylnaphth-1-ylcarbonyl, phenylmethylcarbonyl, 2-methylphenylmethylcarbonyl, 3-methylphenylmethylcarbonyl, 4-methylphenylmethylcarbonyl, 2-methoxyphenylmethylcarbonyl, 4-methoxyphenylmethylcarbonyl, 3-methoxyphenylmethylcarbonyl, 2-chlorophenylmethylcarbonyl, 3-chlorophenylmethylcarbonyl, 4-chlorophenylmethylcarbonyl, 2-bromophenylmethylcarbonyl, morphlinylmethylcarbonyl, 2,2,4-trimethyl-3-bicyclo[2.2.1]heptylaminocarbonyl, 1-pyrollidinylcarbonyl, 4-benzylpiperazin-1-ylcarbonyl, 3-pyridylcarbonyl, quinolin-3-yloxycarbonyl, quinolin-4-yloxycarbonyl, quinolin-5-yloxycarbonyl, quinolin-6-yloxycarbonyl, quinolin-7-yloxycarbonyl, quinolin-8-yloxycarbonyl, 4-isoquinolinyloxycarbonyl, 5-isoquinolinyloxycarbonyl, 6-isoquinolinyloxycarbonyl, 7-isoquinolinyloxycarbonyl, 8-isoquinolinyloxycarbonyl, 2,2,3,3-tetramethylcyclopropylcarbonyl, adamantylcarbonyl, 4-methylpiperazin-1-ylcarbonyl, 3,4-dimethylpiperazin-1-ylcarbonyl, phenylaminocarbonyl, phenylmethylaminocarbonyl, phenyl-(1,1-dimethylmethyl)aminocarbonyl, 1-naphthylaminocarbonyl, adamantylaminocarbonyl, cyclopropylaminocarbonyl, 2,2,4-trimethyl-3-bicyclo[2.2.1]heptanylaminocarbonyl, naphthylmethyl, and morpholinylcarbonylmethyl; R⁵ is H, methyl or ethyl; and R⁶ is H, bromo, nitro, lodo, methoxy or 4-methylnaphtyl; or pharmaceutically acceptable salt thereof.

In some embodiments, the method comprises compounds of Formula II wherein R¹ is selected from butyl, pentyl, 2-methylpropyl, 2,2-dimethylpropyl, 2-methylbutyl, 1-ethylpropyl, 3-chloropentyl, 3-fluoropentyl, 5-fluoropentyl, 5-bromopentyl, cyclohexylmethyl, cyclohexylethyl, 1-methyl-azepan-3-yl, 4-morpholinylethyl, 4-fluorophenylmethyl and 1-methylpiperidin-2-ylmethyl; R² is aminocarbonyl-(2,2-dimethylpropyl)aminocarbonyl, methoxycarbonyl-(2,2-dimethylpropyl)aminocarbonyl, 2-iodo-5-nitophenylcarbonyl, 2-methoxyphenylcarbonyl, 2-iodophenylcarbonyl, 1-naphthylcarbonyl, 2-naphthylcarbonyl, 1-methoxy-4-naphthylcarbonyl, 2-methyl-1-naphthylcarbonyl, 8-chloro-1-naphthylcarbonyl, 4-methoxyphenylmethylcarbonyl, 3-methoxyphenylmethylcarbonyl, 2,2,4-trimethyl-3-bicyclo[2.2.1]heptylaminocarbonyl, quinolin-4-yloxycarbonyl, quinolin-6-yloxycarbonyl, 4-isoquinolinyloxycarbonyl, 7-isoquinolinyloxycarbonyl, quinolin-6-yloxycarbonyl, quinolin-7-yloxycarbonyl, quinolin-8-yloxycarbonyl, 2,2,3,3-tetramethylcyclopropylcarbonyl, and 8-isoquinolinyloxycarbonyl, R⁵ is H; and R⁶ is H or methoxy; or pharmaceutically acceptable salt thereof.

In one aspect, the invention provides a method wherein the cannabinoid derivative is a compound of Formula III

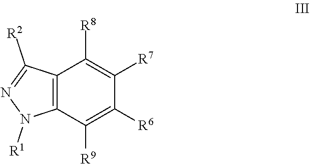

wherein R¹ is selected from alkyl, haloalkyl, cyanoalkyl, cycloalkylalkyl, heterocyclylalkyl, arylalkyl and aryl; R² is aminocarbonyl(alkyl)aminocarbonyl, alkoxycarbonyl(alkyl)aminocarbonyl, aminocarbonyl(arylalkyl)aminocarbonyl, arylcarbonyl, heterocyclylcarbonyl, aralkylcarbonyl, aryloxycarbonyl, alkoxycarbonyl(alkyl)oxycarbonyl, cycloalkyloxycarbonyl, heterocyclyloxycarbonyl, arylalkylaminocarbonyl, heterocyclylaminocarbonyl, and cycloalkylcarbonyl; R⁶ is H; R⁷ is H; R⁸ is H; and R⁹ is H; or pharmaceutically acceptable salt thereof

In some embodiments, the method comprises compounds of Formula III wherein R¹ is selected from C₁₋₈-alkyl, C₁₋₆-haloalkyl, C₁₋₆-cyanoalkyl, C₃₋₈-cycloalkyl-C₁₋₆-alkyl, 3-8 membered heterocyclyl-C₁₋₆ alkyl, optionally substituted phenyl-C₁₋₆-alkyl and optionally substituted phenyl; and R² is aminocarbonyl(C₁₋₆-alkyl)aminocarbonyl, C₁₋₆-alkoxycarbonyl(C₁₋₆-alkyl)aminocarbonyl, aminocarbonyl(optionally substituted phenyl-C₁₋₆-alkyl)aminocarbonyl, optionally substituted C₆₋₁₀-arylcarbonyl, 3-10 membered heterocyclylcarbonyl, optionally substituted phenyl-C₁₋₆-alkylcarbonyl, C₁₋₆-alkoxycarbonyl(C₁₋₆-alkyl)oxycarbonyl, optionally substituted C₆₋₁₀-aryloxycarbonyl, C₃₋₈-cycloalkyloxycarbonyl, 3-10 membered heterocyclyloxycarbonyl, optionally substituted phenyl-C₁₋₆-alkylaminocarbonyl, 3-10 membered heterocyclylaminocarbonyl, and C₃₋₈-cycloalkylcarbonyl; or pharmaceutically acceptable salt thereof.

In some embodiments, the method comprises compounds of Formula III wherein R¹ is selected from pentyl, 2-fluoropentyl, 3-fluoropentyl, 4-fluoropentyl, 5-fluoropentyl, 5-bromopentyl, 5-chloropentyl, 4-cyanobutyl, cyclohexylmethyl, 4-morpholinylmethyl, 2-fluorophenyl, benzyl, 3-fluorobenzyl, 2-fluorobenzyl, and 4-fluorophenylmethyl; and R² is aminocarbonyl-(butyl)aminocarbonyl, aminocarbonyl-(pentyl)aminocarbonyl, aminocarbonyl-(2,2-dimethylpropyl)aminocarbonyl, aminocarbonyl-(1-methylpropyl)aminocarbonyl, aminocarbonyl-(2-methylpropyl)aminocarbonyl, aminocarbonyl-(2-methylbutyl)aminocarbonyl, aminocarbonyl-(3-methylbutyl)aminocarbonyl, aminocarbonyl-(1,1-dimethylpropyl)aminocarbonyl, methoxycarbonyl-(2,2-dimethylethyl)aminocarbonyl, methoxycarbonyl-(2,2-dimethylpropyl)aminocarbonyl, methoxycarbonyl-(1,1-dimethylpropyl)aminocarbonyl, ethoxycarbonyl-(2,2-dimethylethyl)aminocarbonyl, aminocarbonyl(phenylethyl)aminocarbonyl, 1-naphthylcarbonyl, 1-pyrollidinylcarbonyl, phenyl-(1,1-dimethyl)methylcarbonyl, methoxycarbonyl-(2,2-dimethylpropyl)oxycarbonyl, adamantyloxycarbonyl, 1-naphthyloxycarbonyl, 2-naphthyloxycarbonyl, quinolin-8-yloxycarbonyl, 2,2,3,3-tetramethylcyclopropylcarbonyl, 1-naphthylaminocarbonyl, adamantylaminocarbonyl, phenyl-(1,1-dimethyl)methylaminocarbonyl, and 8-quinolinylaminocarbonyl; or pharmaceutically acceptable salt thereof.

In some embodiments, the method comprises compounds of Formula III wherein R¹ is selected from pentyl, cyclohexylmethyl and 4-fluorophenylmethyl; R² is aminocarbonyl-(pentyl)aminocarbonyl, aminocarbonyl-(2-methylbutyl)aminocarbonyl, aminocarbonyl-(3-methylbutyl)aminocarbonyl, methoxycarbonyl-(2,2-dimethylethyl)aminocarbonyl, methoxycarbonyl-(1,1-dimethylpropyl)aminocarbonyl, methoxycarbonyl-(2,2-dimethylpropyl)oxycarbonyl, aminocarbonyl(phenylethyl)aminocarbonyl and ethoxycarbonyl-(2,2-dimethylethyl)aminocarbonyl; or pharmaceutically acceptable salt thereof.

In one aspect, the invention provides a method wherein the cannabinoid derivative is a compound of Formula IV

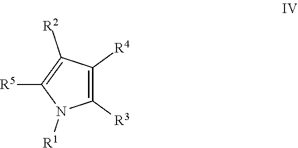

Wherein R¹ is alkyl or arylalkyl; R² is H or arylcarbonyl; R³ is H or aryl; R⁴ is H or amino; and R⁵ is H, C₁₋₆-alkyl or arylcarbonyl; or pharmaceutically acceptable salt thereof.

In some embodiments, the method comprises compounds of Formula IV wherein R¹ is C₁₋₆-alkyl or aryl-C₁₋₆-alkyl; R² is H or C₆₋₁₀-arylcarbonyl; R³ is H or optionally substituted C₆₋₁₀-aryl; R⁴ is H or amino; and R⁵ is H, C₁₋₆-alkyl or C₆₋₁₀-arylcarbonyl; or pharmaceutically acceptable salt thereof.

In some embodiments, the method comprises compounds of Formula IV wherein R¹ is pentyl, hexyl, heptyl or 4-chlorophenylmethyl; R² is H, phenylcarbonyl or naphthylcarbonyl; R³ is H, phenyl, 2-methylphenyl, 2-fluorophenyl, 2-chlorophenyl, 3-fluorophenyl, or naphthyl; R⁴ is H or amino; and R⁵ is H, methyl or naphthylcarbonyl; or pharmaceutically acceptable salt thereof.

In some embodiments, the method comprises compounds of Formula IV wherein R² is arylcarbonyl when R⁵ is H or alkyl; or pharmaceutically acceptable salt thereof.

In one aspect, the invention provides a method wherein the cannabinoid derivative is a compound of Formula V

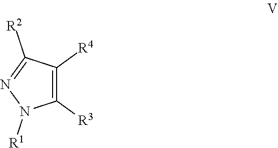

Wherein R¹ is haloalkyl or cycloalkylalkyl; R² is aminocarbonyl(alkyl)aminocarbonyl or optionally substituted aryl; R³ is optionally substituted aryl; and R⁴ is H; or pharmaceutically acceptable salt thereof.

In some embodiments, the method comprises compounds of Formula V wherein R¹ is C₁₋₆-haloalkyl or C₃₋₆-cycloalkyl-C₁₋₆-alkyl; R² is aminocarbonyl-(C₁₋₆-alkyl)aminocarbonyl or optionally substituted phenyl; R³ is optionally substituted phenyl or aminocarbonyl-(C₁₋₆-alkyl)aminocarbonyl; and R⁴ is H; or pharmaceutically acceptable salt thereof. In some embodiments, the method comprises compounds of Formula V wherein R¹ is 5-fluoropentyl or cyclohexylmethyl; R² is aminocarbonyl-(1,1-dimethylethyl)aminocarbonyl or 4-fluorophenyl; R³ is aminocarbonyl-(2,2-dimethylpropyl)aminocarbonyl or aminocarbonyl-(1,1-dimethylethyl)aminocarbonyl or 4-fluorophenyl; and R⁴ is H; or pharmaceutically acceptable salt thereof.

In some embodiments, the method comprises compounds of Formula V wherein R² is aminocarbonyl-(1,1-dimethylethyl)aminocarbonyl when R³ is 4-fluorophenyl; or pharmaceutically acceptable salt thereof.

In one aspect, the invention provides a method wherein the cannabinoid derivative is a compound of Formula VI

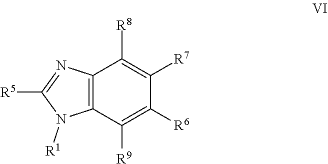

Wherein R¹ is alkyl, haloalkyl or cycloalkylalkyl; R⁵ is arylcarbonyl, aminocarbonylalkylaminocarbonyl or optionally substituted arylalkyl; R⁶ is H; R⁷ is H; R⁸ is H; and R⁹ is H or alkylaminocarbonyl; or pharmaceutically acceptable salt thereof.

In some embodiments, the method comprises compounds of Formula VI wherein R¹ is C₁₋₆-alkyl, C₁₋₆-haloalkyl or cyclohexyl-C₁₋₆-alkyl; R⁵ is C₆₋₁₀-aryl-C₁₋₆-alkylcarbonyl, aminocarbonyl-C₁₋₆-alkylaminocarbonyl or substituted phenyl-C₁₋₆-alkyl; and R⁶ is H or C₁₋₆-alkylaminocarbonyl; or pharmaceutically acceptable salt thereof.

In some embodiments, the method comprises compounds of Formula VI wherein R¹ is pentyl, 5-fluoropentyl, 2-hydroxy-2methylbutyl or cyclohexylmethyl; R⁵ is naphthylcarbonyl, aminocarbonyl-(1,1-di-methylpropyl)aminocarbonyl or 4-ethoxyphenylmethyl; and R⁶ is H or diethylaminocarbonyl; or pharmaceutically acceptable salt thereof.

In some embodiments, the method comprises compounds of Formula VI wherein R⁵ is optionally substituted arylalkyl when R⁶ is alkylaminocarbonyl; or pharmaceutically acceptable salt thereof.

In one aspect, the invention provides a method wherein the cannabinoid derivative is a compound of Formula VII

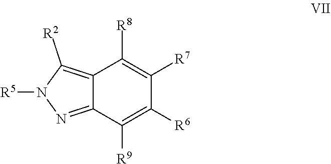

Wherein R² is arylaminocarbonyl, aminocarbonylalkylaminocarbonyl or arylalkylcarbonyl; R⁵ is haloalkyl, cycloalkylalkyl, alkyl or cyanoalkyl; R⁶ is H; R⁷ is H; R⁸ is H; and R⁹ is H; or pharmaceutically acceptable salt thereof.

In some embodiments, the method comprises compounds of Formula VII wherein R² is arylaminocarbonyl, aminocarbonyl-C₁₋₆-alkylaminocarbonyl or aryl-C₁₋₆-alkylcarbonyl; and R⁵ is C₁₋₆-haloalkyl, cyclohexyl-C₁₋₆-alkyl, C₁₋₆-alkyl or cyano-C₁₋₆-alkyl; or pharmaceutically acceptable salt thereof.

In some embodiments, the method comprises compounds of Formula VII wherein R² is naphthylaminocarbonyl, aminocarbonyl-(2-methylbutyl)aminocarbonyl or 1,1-dimethylphenylmethylcarbonyl; and R⁵ is 5-fluoropentyl, cyclohexylmethyl, pentyl or 4-cyanobutyl; or pharmaceutically acceptable salt thereof.

In one aspect, the invention provides a method wherein the cannabinoid derivative is a compound of Formula VIII

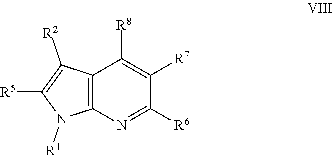

Wherein R¹ is haloalkyl; R² is arylalkylcarbonyl; R⁵ is H; R⁶ is H; R⁷ is H; and R⁸ is H; or pharmaceutically acceptable salt thereof.

In some embodiments, the method comprises compounds of Formula VIII wherein R¹ is C₁₋₆-haloalkyl; and R² is aryl-C₁₋₆-alkylcarbonyl; or pharmaceutically acceptable salt thereof.

In some embodiments, the method comprises compounds of Formula VIII wherein R¹ is 5-fluoropentyl; and R² is 1,1-dimethylphenylmethylcarbonyl; or pharmaceutically acceptable salt thereof.

Numerous embodiments of the compounds of Formula I-VIII are set forth herein. It is understood that any of the foregoing disclosed compounds may be used in any and all aspects or embodiments of the invention described herein.

Treatment in accordance with the present invention may be symptomatic or prophylactic. The compounds of the current invention can be administered at three general times: [1] prophylactic or before virus infection, [2] after virus infection but before occurrence of symptoms or while asymptomatic, and [3] after occurrence of symptoms.

Cannabinoids have demonstrated biological and pharmacological effects, including pain reduction, inhibition of nausea, appetite induction, anxiety and depression reduction, and anticonvulsant, among others (Robson P, Handbook Exp Pharmacol 168:719-756, 2005).

The method of the invention is of general application to mammalian cells, including virally infected cells.

Combining pharmaceutical compositions of the invention with other therapeutics, particularly antiviral compounds or vaccines may further increase the potency of the cannabinoid agents. Appropriate doses are determined by the condition of the patient and degree of severity of the disorder under treatment, but are within the ordinary skill of the attending clinician based upon analogy with other virus treating pharmaceuticals.

When administered as a combination, the therapeutic agents can be formulated as separate compositions that are administered at the same time or sequentially at different times, or the therapeutic agents can be given as a single composition. The phrase “co-therapy” (or “combination-therapy”), in defining use of a compound of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent. Specifically, the administration of compounds of the present invention may be in conjunction with additional therapies known to those skilled in the art.

If formulated as a fixed dose, such combination products employ the compounds of this invention within the accepted dosage ranges. Compounds of any of the embodiments described herein may also be administered sequentially with known agents for use in treating viral infections, when a combination formulation is inappropriate. The invention is not limited in the sequence of administration as compounds of the invention may be administered either prior to, simultaneous with, or after administration of one or more additional therapeutic agents.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound. The compounds of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

The term “comprising” is meant to be open ended, i.e., all-encompassing and non-limiting. It may be used herein synonymously with “having” or “including”. Comprising is intended to include each and every indicated or recited component or element(s) while not excluding any other components or elements. For example, if a composition is said to comprise A and B, this means that the composition has A and B in it, but may also include C or even C, D, E, and other additional components.

Certain compounds of the invention may possess asymmetric carbon atoms (optical centers) or double bonds; the racemates, enantiomers, diastereomers, geometric isomers and individual isomers are all intended to be encompassed within the scope of the invention. Furthermore, atropisomers and mixtures thereof such as those resulting from restricted rotation about two aromatic or heteroaromatic rings bonded to one another are intended to be encompassed within the scope of the invention. For example, when R4 is a phenyl group and is substituted with two groups bonded to the C atoms adjacent to the point of attachment to the N atom of the triazole, then rotation of the phenyl may be restricted. In some instances, the barrier of rotation is high enough that the different atropisomers may be separated and isolated.

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the mirror image enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. If the stereochemistry of a structure or a portion of a structure is not indicated with, for example, bold or dashed lines, the structure or portion of the structure is to be interpreted as encompassing all stereoisomers of it. A bond drawn with a wavy line indicates that both stereoisomers are encompassed. This is not to be confused with a wavy line drawn perpendicular to a bond which indicates the point of attachment of a group to the rest of the molecule.

As described above, this invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al. (1997) Tetrahedron 33:2725; Eliel, E. L., Stereochemistry of Carbon Compounds (McGraw-Hill, N Y, 1962); and Wilen, S. H., Tables of Resolving Agents and Optical Resolutions p.268 (E. L. Eliel, Ed., Univ. of Notre Dame Press, Notre Dame, Ind., 1972).

As known by those skilled in the art, certain compounds of the invention may exist in one or more tautomeric forms. Because one chemical structure may only be used to represent one tautomeric form, it will be understood that for convenience, referral to a compound of a given structural formula includes tautomers of the structure represented by the structural formula.

Compounds of the invention may be depicted structurally and named as compounds in one form. However, it is specifically contemplated and known that the compounds exist in other forms and thus compounds in all tautomeric forms are expressly considered to be part of the invention. Depending on the compound, some compounds may exist primarily in one form more than another.

Compounds of the present disclosure include, but are not limited to, compounds of Formula I-VIII and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. As used herein, the term “compound” encompasses not only the compound itself, but also a pharmaceutically acceptable salt thereof, a solvate thereof, a chelate thereof, a non-covalent complex thereof, a prodrug thereof, and mixtures of any of the foregoing. In some embodiments, the term “compound” encompasses the compound itself, pharmaceutically acceptable salts thereof, tautomers of the compound, pharmaceutically acceptable salts of the tautomers, and ester prodrugs such as (C₁-C₄)alkyl esters. In other embodiments, the term “compound” encompasses the compound itself, pharmaceutically acceptable salts thereof, tautomers of the compound, pharmaceutically acceptable salts of the tautomers.

The term “solvate” refers to the compound formed by the interaction of a solvent and a compound. Suitable solvates are pharmaceutically acceptable solvates, such as hydrates, including monohydrates and hemi-hydrates.

Disease” refers to any disease, disorder, condition, symptom, or indication.

The term “cannabinoid compound” means a compound that can be isolated from cannabis, also known as “phytocannabinoid”, or an endocannabinoid. The term “cannabinoid compound” also means a synthetic compound that stimulates or inhibits cannabinoid receptors CB-1 and/or CB-2. Such synthetic compounds can also be described as “cannabinoid mimetics”. The term “endocannabinoid” means any compound or metabolite naturally occurring in vertebrates that stimulates or inhibits cannabinoid receptors CB-1 and/or CB-2. The term “synthetic compound” means all chemically synthesized compounds and all non-naturally occurring compounds. Synthetic cannabinoids include the indoles described in U.S. Pat. No. 6,900,236, or U.S. Pat. No. 6,013,648 and the pyrazoles in U.S. Pat. No. 7,119,108. Cannabinoid derivatives and mimetics include the alkyl-modified compounds described in U.S. Pat. Nos. 9,611,213, 9,517,989, and 10,004,722, as well as the compounds in U.S. Pat. Nos. 6,545,041, 6,903,137, US20020072539, U.S. Ser. No. 10/441,553, U.S. Ser. No. 10/239,848, U.S. Pat. Nos. 9,428,431, 8,497,299, 7,884,133, US20100152238, U.S. Pat. Nos. 4,876,276, 5,521,215, 5,538,993, 6,096,740, 5,284,867, 5,635,530, 4,179,517, US20200115317 Δ1 and WO2008107879 “Cannabinoid compound” further means a compound that stimulates or inhibits cannabinoid receptors CB-1 and/or CB-2. Cannabinoids have been described in Jung et al., Chem. Asian J. 2019, 14, 3749-3762, Hanus et al., Nat Prod Rep, 2016, 33, 1357-1448, Shevyrin et al., Russ Chem Bull, Intern Ed, 2015, 64, 1249-1266, and Lewis et al., ACS Omega 2017 2, 6091-6103.

Phytocannabinoids include tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV),_cannabichromevarin(CBCV), cannabigerovarin (CBGV), cannabigerol monomethyl ether (CBGM), cannabielsoin (CBE), and cannabicitran (CBT).

The term “derivative” used herein shall include any conventionally known derivatives of the cannabinoid, such as, inter alia, solvates. It may be convenient or desirable to prepare, purify, and/or handle a corresponding solvate of the compound described herein, which may be used in any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc., depending on the number of water molecules present per molecule of substrate. The term derivative shall especially include a salt. Suitable salts of the cannabinoid are well known and are described in the prior art. Salts of organic and inorganic acids and bases may be used to make pharmaceutically acceptable salts. Such acids include, without limitation, hydrofluoric, hydrochloric, hydrobromic, hydroiodic, sulfuric, nitric, phosphoric, citric, succinic, maleic, and palmitic acids. The bases include such compounds as sodium and ammonium hydroxides. Those skilled in the art are familiar with quaternizing agents that can be used to make pharmaceutically acceptable quaternary ammonium derivatives of the cannabinoid. These include without limitation methyl and ethyl iodides and sulphates.

“Alkyl” refers to a saturated branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl and propan-2-yl, butyls such as butan-1-yl, butan-2-yl, 2-methyl-propan-1-yl, 2-methyl-propan-2-yl, tert-butyl, and the like. In certain embodiments, an alkyl group comprises 1 to 20 carbon atoms. In some embodiments, alkyl groups include 1 to 8 carbon atoms or 1 to 6 carbon atoms whereas in other embodiments, alkyl groups include 1 to 4 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl group and an alkyl group having 1 to 4 carbon atoms may be referred to as a (C₁-C₄)alkyl. This nomenclature may also be used for alkyl groups with differing numbers of carbon atoms. The term “alkyl may also be used when an alkyl group is a substituent that is further substituted in which case a bond between a second hydrogen atom and a C atom of the alkyl substituent is replaced with a bond to another atom such as, but not limited to, a halogen, or an O, N, or S atom. For example, a group-O—(C₁-C₆ alkyl)-OH will be recognized as a group where an —O atom is bonded to a C₁-₆ alkyl group and one of the H atoms bonded to a C atom of the C₁-₆ alkyl group is replaced with a bond to the 0 atom of an—OH group. As another example, a group-O—(C₁-C₆ alkyl)-O—(C₁-C₆ alkyl) will be recognized as a group where an —O atom is bonded to a first C₁-C₆ alkyl group and one of the H atoms bonded to a C atom of the first C₁-C₆ alkyl group is replaced with a bond to a second 0 atom that is bonded to a second C₁-C₆ alkyl group.

“Alkenyl” refers to an unsaturated branched or straight-chain hydrocarbon group having at least one carbon-carbon double bond derived by the removal of one hydrogen atom from a single carbon atom of a parent alkene. The group may be in either the Z- or E- form (cis or trans) about the double bond(s). Typical alkenyl groups include, but are not limited to, ethenyl; propenyls such as prop-1-en-1-yl, prop-1-en-2-yl, prop-2-en-1-yl (allyl), and prop-2-en-2-yl; butenyls such as but-1-en-1-yl, but-1-en-2-yl, 2-methyl-prop-1-en-1-yl, but-2-en-1-yi, but-2-en-1-yl, but-2-en-2-yl, buta-1,3-dien-1-yl, and buta-1,3-dien-2-yl; and the like. In certain embodiments, an alkenyl group has 2 to 20 carbon atoms and in other embodiments, has 2 to 6 carbon atoms. An alkenyl group having 2 to 6 carbon atoms may be referred to as a (C₂—Cc)alkenyl group.

“Alkoxy” refers to a radical-OR where R represents an alkyl group as defined herein. Representative examples include, but are not limited to, methoxy, ethoxy, propoxy, butoxy, and the like. Typical alkoxy groups include 1 to 10 carbon atoms, 1 to 6 carbon atoms or 1 to 4 carbon atoms in the R group. Alkoxy groups that include 1 to 6 carbon atoms may be designated as—O—(C₁-C₆) alkyl or as—O—(C₁-C₆ alkyl) groups. In some embodiments, an alkoxy group may include 1 to 4 carbon atoms and may be designated as—O—(C₁-C₄) alkyl or as—O—(C₁-C₄ alkyl) groups group.

“Aryl” refers to a monovalent aromatic hydrocarbon group derived by the removal of one hydrogen atom from a single carbon atom of a parent aromatic ring system. Aryl encompasses monocyclic carbocyclic aromatic rings, for example, benzene. Aryl also encompasses bicyclic carbocyclic aromatic ring systems where each of the rings is aromatic, for example, naphthalene. Aryl groups may thus include fused ring systems where each ring is a carbocyclic aromatic ring. In certain embodiments, an aryl group includes 6 to 10 carbon atoms. Such groups may be referred to as C₈-C₁₀ aryl groups. Aryl, however, does not encompass or overlap in any way with heteroaryl as separately defined below. Hence, if one or more carbocyclic aromatic rings is fused with an aromatic ring that includes at least one heteroatom, the resulting ring system is a heteroaryl group, not an aryl group, as defined herein. Said “aryl” group may have 1 to 3 substituents such as lower alkyl, hydroxy, halo, haloalkyl, nitro, cyano, alkoxy and lower alkylamino.

The term “aralkyl” embraces aryl-substituted alkyl radicals. Preferable aralkyl radicals are “lower aralkyl” radicals having aryl radicals attached to alkyl radicals having one to six carbon atoms. Examples of such radicals include benzyl, diphenylmethyl and phenylethyl. The aryl in said aralkyl may be additionally substituted with halo, alkyl, alkoxy, halkoalkyl and haloalkoxy.

The term “aryloxy” embraces aryl radicals, as defined above, attached to an oxygen atom. Examples of such radicals include phenoxy and naphthyloxy.

The term “aryloxycarbonyl” embraces aryloxy radicals, as defined above, attached to carbonyl radical. Examples of such radicals include naphthyloxycarbonyl.

“Carbonyl” refers to the radical-C(O) which may also be referred to as —C(═O) group.

“Cyano” refers to the radical —CN.

The term “cyanoalkyl” embraces linear or branched alkyl radicals having one to about ten carbon atoms any one of which may be substituted with one cyano radicals. More preferred cyanoalkyl radicals are “lower cyanoalkyl” radicals having one to six carbon atoms and one cyano radical. Examples of such radicals include cyanobutyl.

“Cycloalkyl” refers to a saturated cyclic alkyl group derived by the removal of one hydrogen atom from a single carbon atom of a parent cycloalkane. Typical cycloalkyl groups include, but are not limited to, groups derived from cyclopropane, cyclobutane, cyclopentane, cyclohexane, cycloheptane, cyclooctane, and the like. Cycloalkyl groups may be described by the number of carbon atoms in the ring. For example, a cycloalkyl group having 3 to 8 ring members may be referred to as a (C₃-C₈)cycloalkyl, a cycloalkyl group having 3 to 7 ring members may be referred to as a (C3-C₇)cycloalkyl and a cycloalkyl group having 4 to 7 ring members may be referred to as a (C₄-C₇)cycloalkyl. In certain embodiments, the cycloalkyl group can be a (C₃-C₁₀)cycloalkyl, a (C3-C₇)cycloalkyl, a (C3-C1)cycloalkyl, a (C3-Cr)cycloalkyl, or a (C₄-C₇)cycloalkyl group and these may be referred to as C₃-C₁₀ cycloalkyl, C₃-C₈ cycloalkyl, C₃-C₇ cycloalkyl, C₃-C₆ cycloalkyl, or C₄-C₇ cycloalkyl groups using alternative language.

The term “cycloalkylalkyl”, embraces radicals having a cycloalkyl radical as defined above, attached to an alkyl radical. Examples of such cycloalkylalkyl radicals includes cyclohexylmethyl.

“Heterocyclyl” refers to a cyclic group that includes at least one saturated, partially unsaturated, but non-aromatic, cyclic ring. Heterocyclyl groups include at least one heteroatomn as a ring member. Typical heteroatoms include, O, S and N and are independently chosen. Heterocyclyl groups include monocyclic ring systems and bicyclic ring systems. Bicyclic heterocyclyl groups include at least one non-aromatic ring with at least one heteroatom ring member that may be fused to a cycloalkyl ring or may be fused to an aromatic ring where the aromatic ring may be carbocyclic or may include one or more heteroatoms. The point of attachment of a bicyclic heterocyclyl group may be at the non-aromatic cyclic ring that includes at least one heteroatom or at another ring of the heterocyclyl group. For example, a heterocyclyl group derived by removal of a hydrogen atom from one of the 9 membered heterocyclic compounds shown below may be attached to the rest of the molecule at the 5-membered ring or at the 6-membered ring. In some embodiments, a heterocyclyl group includes 5 to 10 ring members of which 1, 2, 3 or 4 or 1, 2, or 3 are heteroatoms independently selected from O, S, or N. In other embodiments, a heterocyclyl group includes 3 to 7 ring members of which 1, 2, or 3 heteroatom are independently selected from O, S, or N. In such 3-7 membered heterocyclyl groups, only 1 of the ring atoms is a heteroatom when the ring includes only 3 members and includes 1 or 2 heteroatoms when the ring includes 4 members. In some embodiments, a heterocyclyl group includes 3 or 4 ring members of which 1 is a heteroatom selected from O, S, or N. In other embodiments, a heterocyclyl group includes 5 to 7 ring members of which 1, 2, or 3 are heteroatoms independently selected from O, S, or N. Typical heterocyclyl groups include, but are not limited to, groups derived from epoxides, aziridine, azetidine, imidazolidine, morpholine, piperazine, piperidine, hexahydropyrimidine, 1,4,5,6-tetrahydropyrimidine, pyrazolidine, pyrrolidine, quinuclidine, tetrahydrofuran, tetrahydropyran, benzimidazolone, pyridinone, and the like. Heterocyclyl groups may be fully saturated, but may also include one or more double bonds. Examples of such heterocyclyl groups include, but are not limited to, 1,2,3,6-tetrahydropyridinyl, 3,6-dihydro-2H-pyranyl, 3,4-dihydro-2H-pyranyl, 2,5-dihydro-1 H-pyrolyl, 2,3-dihydro-1 H-pyrolyl, 1 H-azirinyl, 1,2-dihydroazetenyl, and the like. Substituted heterocyclyl also includes ring systems substituted with one or more oxo (═O) or oxide (—O—) substituents, such as piperidinyl N-oxide, morpholinyl-N-oxide, 1-oxo-1-thiomorpholinyl, pyridinonyl, benzimidazolonyl, benzo[d]oxazol-2(3H)-only, 3,4-dihydroisoquinolin-1(2H)-only, indolinonly, 1 H-imidazo[4,5-c]pyridin-2(3H)-only, 7H-purin-8(9H)-only, imidazolidin-2-only, 1 H-imidazol-2(3H)-only, 1,1-dioxo-1-thiomorpholinyl, and the like.

The term “heterocyclylalkyl”, embraces radicals having a heterocyclyl radical as defined above, attached to an alkyl radical. Examples of such heterocyclylalkyl radicals includes piperidinylmethyl.

The term “alkenylcarbonyl” embraces radicals having a carbonyl radical substituted with an alkenyl radical. More preferred alkenylcarbonyl radicals are “lower alkenylcarbonyl” radicals having two to six carbon atoms. Examples of such radicals include ethenylcarbonyl.

The term “arylcarbonyl” embraces radicals having a carbonyl radical substituted with an aryl radical. More preferred arylcarbonyl radicals include phenylcarbonyl.

The term “cycloalkylcarbonyl” embraces radicals having a carbonyl radical substituted with a cycloalklyl radical.

The term “heterocyclylcarbonyl” embraces radicals having a carbonyl radical substituted with a heterocyclyl radical.

The term “arylalkylcarbonyl” embraces radicals having a carbonyl radical substituted with an arylalkyl radical. More preferred arylcarbonyl radicals include benzylcarbonyl.

The term “heterocyclylalkylcarbonyl” embraces radicals having a carbonyl radical substituted with a heterocyclylalkyl radical.

The term “alkoxycarbonyl” means a radical containing an alkoxy radical, as defined above, attached via an oxygen atom to a carbonyl radical, Preferably, “lower alkoxycarbonyl” embraces alkoxy radicals having one to six carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl.

The term “aminocarbonyl” when used by itself or with other terms such as “aminocarbonylalkyl”, “N-alkylaminocarbonyl”, “N-arylaminocarbonyl”, and “N,N-dialkylaminocarbonyl”, denotes an amide group of the formula —C(═O)NH₂. The terms “N-alkylaminocarbonyl” and “N,N-dialkylaminocarbonyl” denote aminocarbonyl radicals which have been substituted with one alkyl radical and with two alkyl radicals, respectively. More preferred are “lower alkylaminocarbonyl” having lower alkyl radicals as described above attached to an aminocarbonyl radical.

The terms “arylaminocarbonyl” and “N-alkyl-N-arylaminocarbonyl” denote aminocarbonyl radicals substituted, respectively, with one aryl radical, or one alkyl and one aryl radical.

The term aminocarbonylalkylaminocarbonyl denotes alkylaminocarbonyl radicals substituted with one aminocarbonyl radical.

The term alkoxycarbonylalkylaminocarbonyl, denotes alkylaminocarbonyl radicals substituted with one alkoxycarbonyl radical.

The term aminocarbonyl(arylalkyl)aminocarbonyl, denotes arylalkylaminocarbonyl radicals substituted with one aminocarbonyl radical.

The term “N-cycloalkylaminocarbonyl” denoted aminocarbonyl radicals which have been substituted with at least one cycloalkyl radical. More preferred are “lower cycloalkylaminocarbonyl” having lower cycloalkyl radicals of three to seven carbon atoms, attached to an aminocarbonyl radical.

The terms “heterocyclylaminocarbonyl” denote aminocarbonyl radicals substituted with one heterocyclyl radical.

The term “heterocyclylcarbonylalkyl” embraces radicals having an alkyl substituted with a heterocyclylcarbonyl radical.

“Halo” or “halogen” refers to a fluoro, chloro, bromo, or iodo group.

“Haloalkyl” refers to an alkyl group in which at least one hydrogen is replaced with a halogen. Thus, the term “haloalkyl” includes monohaloalkyl (alkyl substituted with one halogen atom) and polyhaloalkyl (alkyl substituted with two or more halogen atoms). Representative “haloalkyl” groups include difluoromethyl, 2,2,2-trifluoroethyl, 2,2,2-trichloroethyl, and the like. The term “perhaloalkyl” means, unless otherwise stated, an alkyl group in which each of the hydrogen atoms is replaced with a halogen atom. For example, the term “perhaloalkyl”, includes, but is not limited to, trifluoromethyl, pentachloroethyl, 5-fluoropentyl, and the like.

“Heteroaryl” refers to a monovalent heteroaromatic group derived by the removal of one hydrogen atom from a single atom of a parent heteroaromatic ring system. Heteroaryl groups typically include 5- to 14-membered, but more typically include 5- to 10-membered aromatic, monocyclic, bicyclic, and tricyclic rings containing one or more, for example, 1, 2, 3, or 4, or in certain embodiments, 1, 2, or 3, heteroatoms chosen from O, S, or N, with the remaining ring atoms being carbon. In monocyclic heteroaryl groups, the single ring is aromatic and includes at least one heteroatom. In some embodiments, a monocyclic heteroaryl group may include 5 or 6 ring members and may include 1, 2, 3, or 4 heteroatoms, 1, 2, or 3 heteroatoms, 1 or 2 heteroatoms, or 1 heteroatom where the heteroatom(s) are independently selected from O, S, or N. In bicyclic aromatic rings, both rings are aromatic. In bicyclic heteroaryl groups, at least one of the rings must include a heteroatom, but it is not necessary that both rings include a heteroatom although it is permitted for them to do so. For example, the term “heteroaryl” includes a 5- to 7-membered heteroaromatic ring fused to a carbocyclic aromatic ring or fused to another heteroaromatic ring. In tricyclic aromatic rings, all three of the rings are aromatic and at least one of the rings includes at least one heteroatom. For fused, bicyclic and tricyclic heteroaryl ring systems where only one of the rings contains one or more heteroatoms, the point of attachment may be at the ring including at least one heteroatom or at a carbocyclic ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Heteroaryl does not encompass or overlap with aryl as defined above. Examples of heteroaryl groups include, but are not limited to, groups derived from acridine, carbazole, cinnoline, furan, imidazole, indazole, indole, indolizine, isobenzofuran, isochromene, isoindole, isoquinoline, isothiazole, 2H-benzo[d][1,2,3]triazole, isoxazole, naphthyridine, oxadiazole, oxazole, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and the like. In certain embodiments, the heteroaryl group can be between 5 to 20 membered heteroaryl, such as, for example, a 5 to 14 membered or 5 to 10 membered heteroaryl. In certain embodiments, heteroaryl groups can be those derived from thiophene, pyrrole, benzothiophene, 2H-benzo[d][1,2,3]triazole benzofuran, indole, pyridine, quinoline, imidazole, benzimidazole, oxazole, tetrazole, and pyrazine.

The term “heterocyclyloxy” embraces heterocyclyl radicals, as defined above, attached to an oxygen atom. Examples of such radicals include quinolinyloxy.

The term “heterocyclyloxycarbonyl” embraces heterocyclyloxy radicals, as defined above, attached to carbonyl radical. Examples of such radicals include quinolinyloxycarbonyl.

The term “heterocyclylalkyl” embraces heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Examples include such radicals as pyridylmethyl and thienylmethyl.

The term “hydrido” or “H” denotes a single hydrogen atom (H). This hydrido radical may be attached, for example, to an oxygen atom to form a hydroxyl radical or two hydrido radicals may be attached to a carbon atom to form a methylene (—CH₂—) radical.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

“Pharmaceutically acceptable” refers to generally recognized for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Stereoisomer” refers to an isomer that differs in the arrangement of the constituent atoms in space. Stereoisomers that are mirror images of each other and optically active are termed “enantiomers,” and stereoisomers that are not mirror images of one another and are optically active are termed “diastereomers.”

“Subject” includes mammals and humans. The terms “human” and “subject” are used interchangeably herein. Similarly, “patient” is also a subject.

“Therapeutically effective amount” refers to the amount of a compound that, when administered to a subject for treating a disease, or at least one of the clinical symptoms of a disease or disorder, is sufficient to affect such treatment for the disease, disorder, or symptom. As those skilled in the art will recognize. this amount is typically not limited to a single dose, but may comprise multiple dosages over a significant period of time as required to bring about a therapeutic or prophylactic response in the subject. Thus, a “therapeutically effective amount” is not limited to the amount in a single capsule or tablet, but may include more than one capsule or tablet, which is the dose prescribed by a qualified physician or medical care provider. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

Reference is made in detail to embodiments of the present disclosure. While certain embodiments of the present disclosure are described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

In some embodiments, the compound may be any one of these presented above.

In some embodiments, the compound may be any of the compounds shown above or a pharmaceutically acceptable salt thereof.

In still other such embodiments, the embodiment provides any of the compounds shown above, or a pharmaceutically acceptable salt thereof, or a mixture thereof.

In some embodiments, the compound is a salt. Such salts may be anhydrous or associated with water as a hydrate. In some embodiments, the compound may be in a neutral form as a base or an acid.

Also provided are pharmaceutical compositions that include the compound or the pharmaceutically acceptable salt thereof, the tautomer thereof, the pharmaceutically acceptable salt of the tautomer, the stereoisomer of any of the foregoing, or the mixture thereof according to any one of the embodiments and at least one pharmaceutically acceptable excipient, carrier or diluent. In some such embodiments, the compound or the pharmaceutically acceptable salt thereof, the tautomer thereof, the pharmaceutically acceptable salt of the tautomer, the stereoisomer of any of the foregoing, or the mixture thereof according to any one of the embodiments is present in an amount effective for the treatment of a viral infection. In some embodiments, the pharmaceutical composition is formulated for oral delivery whereas in other embodiments, the pharmaceutical composition is formulated for intravenous delivery. In some embodiments, the pharmaceutical composition is formulated for oral administration once a day or QD, and in some such formulations is a unit where the effective amount of the active ingredient ranges from 50 mg to 5000 mg. Alternatively, an oral solution may be provided ranging from a concentration of 1 mg/ml to 50 mg/ml or higher.

One aspect of the invention includes administering a cannabinoid to provide a serum concentration ranging from 0.1 μM to 50 μM. One aspect of the invention includes administering a cannabinoid to provide a serum concentration ranging from 1 μM to 20 μM. One aspect of the invention includes administering a cannabinoid to provide a serum concentration ranging from 5 μM to 20 μM. One aspect of the invention includes administering a cannabinoid to provide a serum concentration of either 10 μM, 20 μM, 5 μM, 1 μM, 15 μM, or 40 μM.

One aspect of the invention includes administering a cannabinoid at a dose of 1 to 100 mg/kg/day, 5-40 mg/kg/day, 10-20 mg/kg/day, 1-2 mg/kg/day, 20-40 mg/kg/day, 45-50 mg/kg/day, 50-60 mg/kg/day, 55-65 mg/kg/day, 60-70 mg/kg/day or 65-75 mg/kg/day.

In some embodiments, the subject is a mammal. In some such embodiments, the mammal is a rodent. In other such embodiments, the mammal is a canine. In still other embodiments, the subject is a primate and, in some such embodiments, is a human.

The pharmaceutical compositions or formulations for the administration of the compounds of this invention may conveniently be presented in unit dosage form and may be prepared by any of the methods well known in the art. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the subject. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

The compounds may also be administered via edible means.

Formulations for oral use may also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil, or coconut oil, or in a mineral oil such as liquid paraffin. The oily suspensions may contain a thickening agent, for example beeswax, hard paraffin, or cetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The pharmaceutical compositions may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include, for example, cocoa butter and polyethylene glycols.

For topical use, creams, ointments, jellies, solutions, or suspensions, etc., containing the compounds of the invention are employed. As used herein, topical application is also meant to include the use of mouthwashes and gargles.

The compounds of the invention can be administered to provide systemic distribution of the compound within the patient. Therefore, in some embodiments, the compounds of the invention are administered to produce a systemic effect in the body.

As indicated above, the compounds of the invention may be administered via oral, mucosal (including sublingual, buccal, rectal, nasal, or vaginal), parenteral (including subcutaneous, intramuscular, bolus injection, intra-arterial, or intravenous), transdermal, or topical administration. In some embodiments, the compounds of the invention are administered via mucosal (including sublingual, buccal, rectal, nasal, or vaginal), parenteral (including subcutaneous, intramuscular, bolus injection, intra-arterial, or intravenous), transdermal, or topical administration. In other embodiments, the compounds of the invention are administered via oral administration. In still other embodiments, the compounds of the invention are not administered via oral administration.

The compound of the invention, the pharmaceutically acceptable salt thereof, the tautomer thereof, the pharmaceutically acceptable salt of the tautomer, the stereoisomer of any of the foregoing, or the mixture thereof may find use in treating a number of conditions. For example, in some embodiments, the invention comprises methods or uses that include the use or administration of the compound, the pharmaceutically acceptable salt thereof, the tautomer thereof, the pharmaceutically acceptable salt of the tautomer, the stereoisomer of any of the foregoing, or the mixture thereof of the invention, in treating a subject suffering from viral infection.

In some embodiments, the compound of the invention, the pharmaceutically acceptable salt thereof, the tautomer thereof, the pharmaceutically acceptable salt of the tautomer, the stereoisomer of any of the foregoing, or the mixture thereof may find use in treating a number of conditions. For example, in some embodiments, the invention comprises methods or uses that include the use or administration of the compound, the pharmaceutically acceptable salt thereof, the tautomer thereof, the pharmaceutically acceptable salt of the tautomer, the stereoisomer of any of the foregoing, or the mixture thereof of the invention, in treating a subject suffering from viral infection.

In some embodiments, the pharmaceutical composition comprises any of the compounds disclosed herein at a purity level suitable for administration to a patient. In some embodiments, the analog has a purity level of at least about 90%, preferably above about 95%, more preferably above about 99%, and a pharmaceutically acceptable diluent, carrier or excipient.

The pharmaceutical compositions may be formulated to achieve a physiologically compatible pH. In some embodiments, the pH of the pharmaceutical composition may be at least 5, or at least 6, or at least 7, depending on the formulation and route of administration.

In various embodiments, single or multiple administrations of the pharmaceutical compositions are administered depending on the dosage and frequency as required and tolerated by the subject. In any event, the composition should provide a sufficient quantity of at least one of the compounds disclosed herein to effectively treat the subject. The dosage can be administered once but may be applied periodically until either a therapeutic result is achieved or until side effects warrant discontinuation of therapy.

The dosing frequency of the administration of the pharmaceutical composition depends on the nature of the therapy and the particular disease being treated. Treatment of a subject with a therapeutically effective amount of a compound, of the invention can include a single treatment or, preferably, can include a series of treatments. In a preferred example, a subject is treated with compound daily, one time per week or biweekly.

Cannabinoids and derivatives thereof are available commercially or can be prepared as described in US patent publications U.S. Pat. Nos. 6,013,648, 4,885,295 or 7,820,144. Alternatively, compounds that interact with and modulate the endogenous cannabinoid receptors, either as agonists, inverse agonists, or antagonists could be prepared as described in Mills et al., Am. J. Med. Sci. 350:59-62, 2015; or De Luca and Fattore, Clin. Thera. 40:1457-1466, 2018.

The invention having been described, the following examples are offered by way of illustration, and not limitation.

### EXAMPLES

### Example 1. Cannabinoids Inhibit Viral Cell Death

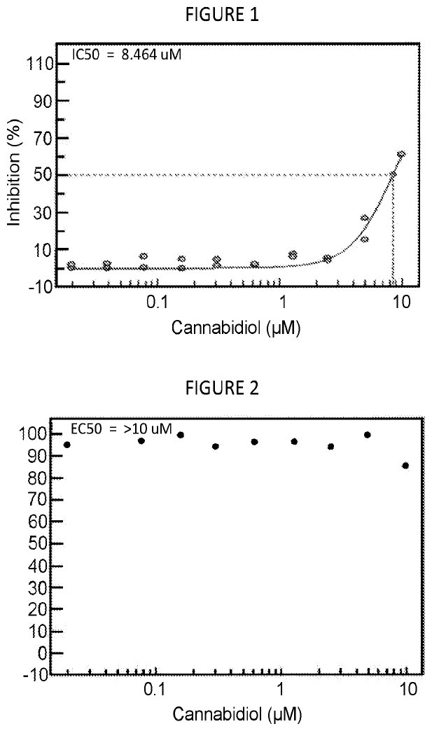

Cannabidiol, a representative cannabinoid, was tested for its ability to inhibit cell toxicity after exposure to SARS-CoV-2 Toronto strain in a cytopathic effect screening assay at Southern Research [Birmingham, Ala.]. To determine whether a cannabinoid has antiviral activity, Vero E6 cells were mixed with virus and then CBD was added at concentrations ranging from 20 nM to 10 μM CBD. Cell viability was determined after 72 hours. As control for non-specific compound toxicity, an identical experiment was conducted with Vero E6 cultures not exposed to virus. As shown in FIG. 1, CBD inhibited cell death of virally-infected cells with an EC₅₀ of 8.5 μM. No cytotoxicity of cannabidiol was observed over the concentration range tested.

### Example 2

### Cannabinoids Inhibit Viral Infection in Human Lung Epithelial Adenocarcinoma Cells

The compounds of the present invention were tested for their ability to inhibit cell toxicity after exposure to SARS-CoV-2 Everett strain in a human lung epithelial adenocarcinoma cell line Calu-3. Calu-3 cells were challenged with SARS2 by the following method. Cells were seeded into 384 well plates at a density of 1,000 cells/well, in 50 μL complete medium (EMEM, 20% FBS, pen/strep, 5 mM HEPES), and allowed 48 hours to recover prior to compound addition and virus infection. The compounds (CBD, cannabidivarin, cannabigerol and a co-treatment with cannabidiol and remdesivir) were diluted into 100% DMSO and 5 nL added to each well with calibrated slotted pins. Assays were performed in duplicate of eight three-fold serial dilutions from a starting concentration of 10 μM. Virus was added within 2 hours after compound addition. The virus was diluted in EMEM, with 5 μL added to each well. The multiplicity of infection was 0.5 to 2.0. The infected cells were incubated for 96 hours at 37° C. in 5% CO2. Viability was assessed with CellTiter Glo 2.0 (Promega) using manufacturer recommended procedures. Data analysis was performed with MS Excel and the add-in XLfit (IDBS). All data were fitted with a 4 Parameter Logistic Dose Response Model. Each plate had controls (blank, negative with no virus, and positive with virus). For the co-treatment with cannabidiol and remdesivir, cultures were exposed to a fixed ratio of CBD/remdesivir=10/1. Datapoints showing toxicity were excluded from the fit and are labeled with brackets. Wells containing the compound dilutions showed a negative offset in luminescence relative to the positive and negative control wells, resulting in a vertical displacement of the dose response curves. The dynamic range (100%) was preserved. CBD, cannabigerol and cannabidivarin protected Calu-3 cells from SARS2-induced cytotoxicity with IC₅₀s of 120, 120 and 43 nM, respectively. The combination of CBD/remdesivir (10:1) showed an IC₅₀ of 230 nM for CBD. Cytotoxicity of the cannabinoids was observed above 1-10 μM.

### Example 3. Syrian Hamster animal model for SARS/COVID-19

The effect of the compounds of the present invention, such as cannabidiol, on the pathological manifestations of COVID-19 can be assessed using an assay as described by Imai et al, PNAS, 117, 16587-95 (2020) or Chan et al., Clin. Infect. Dis., 71, 2428-46 (2020), each incorporated herein by reference in their entirety and particularly for the assays, materials and methods. Such pathologies include lung weight or chemokine/cytokine levels.

The compounds of the present invention can be evaluated in similar assays as described in Nguyen et al. [bioRx 2021. https://doi.org/10.1101/2021.03.10.432967] and Raj et al. [Int. J Biol. Macromolecules 168 2021 (474-85)].

All of the articles and methods disclosed and claimed herein can be made and executed without undue experimentation in light of the present disclosure. While the articles and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes. The disclosure illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

### EMBODIMENTS

The embodiments listed below are presented in numbered form for convenience and for ease and clarity of reference in referring back to multiple embodiments. The embodiments include:

1. A method of treating a subject infected or suspected of being infected with an RNA virus, comprising administering to the subject a cannabinoid compound, or derivative thereof; provided the RNA virus is not hepatitis or influenza.
2. The method of claim 1 wherein the RNA virus is (+)ssRNA virus.
3. The method of Claim 1 wherein the RNA virus is coronavirus.
4. The method of Claim 1 wherein the RNA virus is selected from human coronavirus OC43, human coronavirus HKU1, human coronavirus 229E, human coronavirus NL-63, Middle East respiratory syndrome coronavirus, Severe acute respiratory syndrome coronavirus and Severe acute respiratory syndrome coronavirus 2.
5. The method of any one of claims 1-4 wherein the cannabinoid compound or derivative thereof is a compound of Table 1.
6. The method of any one of claims 1-4 wherein the cannabinoid compound or derivative thereof is a compound of Table 2.
7. The method of any one of claims 1-4 wherein the cannabinoid compound or derivative thereof is a phytocannabinoid.
8. The method of any one of claims 1-4 wherein the cannabinoid compound or derivative thereof is selected from cannabigerol, cannabidiol, cannabichromene, cannabinol, tetrahydrocannabivarin, cannabichromevarin, cannabicitran, Δ8-THC, Δ9-THC, cannabivarin, cannabidivarin, CBN Monomethyl Ether, cannabigerorcin, cannabigerorcinic acid and cannabicyclol.
9. The method of any one of claims 1-8 wherein the cannabinoid compound or derivative thereof is cannabidiol.
10. The method of any one of claims 1-9, wherein said method further comprises administering one of more additional therapeutic agent to said subject.
11. The method of claim 10, wherein said additional therapeutic agent(s) is for treating virus-related symptoms.
12. The method of claim 10, wherein said additional therapeutic agent(s) is selected from steroids, zinc supplements, nucleoside analogs, RNA-dependent RNA polymerase inhibitors, reverse transcriptase inhibitors, protease inhibitors, membrane fusion inhibitors, endocytosis inhibitors, autophagy inhibitors, antivirals, antibacterials, antiprotozoals, viral receptor antagonists, vitamin C, nucleotide analogs, antimalarials, ACE inhibitors, ACE2 inhibitors, ACE2 antibodies, recombinant ACE2, MASP-2 antibodies, C₅-antibodies, immunomodulators, IL-1 inhibitors, IL-6 inhibitors, antibiotics, cardiovascular protective agents, anti-coagulants, statins, and pulmonary protective agents.
13. The method of claim 10, wherein said agent(s) is selected from dexamethasone, remdesivir. Iopinavir, ritonavir, indinavir, atazanavir, boceprevir, darunavir, fosamprenavir, nelfinavir, saquinavir, simeprevir, telaprevir, tipranavir, favipiravir, umifenovir, azithromycin, tocilizumab, umifenovir, galidesivir atorvastatin, and Pulmozyme.
14. The method of claim 10 wherein said additional therapeutic agent is an antiviral selected from Abacavir, Aciclovir, Acyclovir, Adefovir, Alisporivir, Amantadine, Amprenavir, Arbidol, Asunaprevir, Atazanavir, Baloxavir marboxil, Beclabuvir, Boceprevir, Brincidofovir, Brivudine, Cabotegravir, Cidofovir, Cobicistat, Combivir, Daclatasvir, Darunavir, Dasabuvir, Delavirdine, Didanosine, Docosanol, Dolutegravir, Doravirine, Edoxudine, Efavirenz, Elvitegravir, Emtricitabine, Enfuvirtide, Entecavir, Etravirine, Famciclovir, Favipiravir, Filociclovir, Fomivirsen, Fosamprenavir, Foscarnet, Galidesivir, Ganciclovir, Glecaprevir, Grazoprevir, Ibacitabine, Ibalizumab, Idoxuridine, Imiquimod, Imunovir, Indinavir, Interferon alfacon 1, Lamivudine, Laninamivir, octanoate, Letermovir, Lobucavir, Lopinavir, Loviride, Maraviroc, Maribavir, Methisazone, Moroxydine, Nelfinavir, Nevirapine, Nexavir, Nitazoxanide, N-methanocarbathymidine, Norvir, Oseltamivir, Oseltamivir phosphate, Paliviumab, Paritaprevir, Pegylated interferon, Pegylated interferon alfa 2a, Pegylated interferon alfa 2b, Penciclovir, Peramivir, Pibrentasvir, Pimodivir, Pleconaril, Podophyllotoxin, Raltegravir, Recombinant human interleukin-7, Remdesivir, Ribavirin, Rilpivirine, Rimantadine, Rintatolimod, Ritonavir, RSV IGIV, Saquinavir, Simeprevir, Sinecatechins, Sofosbuvir, Stavudine, Taribavirin, Tecovirimat, Telaprevir, Telbivudine, Tenofovir, Tenofovir alafenamide, Tenofovir disoproxil fumarate, Thymalfasin, Tipranavir, Trifluridine, Tromantadine, Umifenovir, Valaciclovir, Valacyclovir, Valganciclovir, Vaniprevir, VariZIG, Vicriviroc, Vidarabine, VZIG, Zalcitabine, Zanamivir, Zidovudine and Zinc gluconate.
15. The method of claim 10 wherein said additional therapeutic agent is selected from Actemra (tocilizumab), sotrovimab, baricitinib, and anti-SARS-CoV-2 monoclonal antibodies such as bamlanivimab, etesevimab, casirivimab, imdevimab, REGN-COV2 ((casirivimab with imdevimab), and regdanvimab and cortisteroids such as prednisone, methylprednisolone,or hydrocortisone.
16. The method of claim 1 wherein the cannabinoid compound, or derivative thereof is administered prior to occurrence of any disease symptoms.
17. The method of claim 1 wherein the cannabinoid compound, or derivative thereof is administered after infection with virus.
18. A method of relieving a symptom of an RNA virus infection in a subject, comprising administering to the subject a cannabinoid compound, or derivative thereof; provided the RNA virus is not hepatitis or influenza.
19. A method of treating an RNA virus-related disease in a subject, comprising administering to the subject a cannabinoid compound, or derivative thereof; provided the RNA virus is not hepatitis or influenza.
20. The method of Claim 17 wherein the disease is COVID-19.
21. A method of providing cytoprotection in a patient in need of such treatment, comprising administering to the patient a pharmacologically effective amount of a cannabinoid.
22. Use of a cannabinoid compound, or derivative thereof; in the manufacture of a medicament for treating an RNA virus, provided the RNA virus is not hepatitis or influenza.

This disclosure includes all modifications and equivalents of the subject matter recited in the aspects appended hereto as permitted by applicable law.

What is claimed is:

1. A method of treating a subject infected or suspected of being infected with an RNA virus, comprising administering to the subject a cannabinoid compound, or derivative thereof; provided the RNA virus is not hepatitis or influenza.

2. The method of claim 1 wherein the RNA virus is (+)ssRNA virus.

3. The method of claim 1 wherein the RNA virus is coronavirus.

4. The method of claim 1 wherein the RNA virus is selected from human coronavirus OC43, human coronavirus HKU1, human coronavirus 229E, human coronavirus NL-63, Middle East respiratory syndrome coronavirus, Severe acute respiratory syndrome coronavirus and Severe acute respiratory syndrome coronavirus 2.

5. The method of any one of claims 1-4 wherein the cannabinoid compound or derivative thereof is a compound of Table 1.

6. The method of any one of claims 1-4 wherein the cannabinoid compound or derivative thereof is a compound of Table 2.

7. The method of any one of claims 1-4 wherein the cannabinoid compound or derivative thereof is a phytocannabinoid.

8. The method of any one of claims 1-4 wherein the cannabinoid compound or derivative thereof is selected from cannabigerol, cannabidiol, cannabichromene, cannabinol, tetrahydrocannabivarin, cannabichromevarin, cannabicitran, Δ8-THC, Δ9-THC, cannabivarin, cannabidivarin, CBN Monomethyl Ether, cannabigerorcin, cannabigerorcinic acid and cannabicyclol.

9. The method of any one of claims 1-8 wherein the cannabinoid compound or derivative thereof is cannabidiol.

10. The method of any one of claims 1-9, wherein said method further comprises administering one of more additional therapeutic agent to said subject.

11. The method of claim 10, wherein said additional therapeutic agent(s) is for treating virus-related symptoms.

12. The method of claim 10, wherein said additional therapeutic agent(s) is selected from steroids, zinc supplements, nucleoside analogs, RNA-dependent RNA polymerase inhibitors, reverse transcriptase inhibitors, protease inhibitors, membrane fusion inhibitors, endocytosis inhibitors, autophagy inhibitors, antivirals, antibacterials, antiprotozoals, viral receptor antagonists, vitamin C, nucleotide analogs, antimalarials, ACE inhibitors, ACE2 inhibitors, ACE2 antibodies, recombinant ACE2, MASP-2 antibodies, C₅-antibodies, immunomodulators, IL-1 inhibitors, IL-6 inhibitors, antibiotics, cardiovascular protective agents, anti-coagulants, statins, and pulmonary protective agents.

13. The method of claim 10, wherein said agent(s) is selected from dexamethasone, remdesivir. lopinavir, ritonavir, indinavir, atazanavir, boceprevir, darunavir, fosamprenavir, nelfinavir, saquinavir, simeprevir, telaprevir, tipranavir, favipiravir, umifenovir, azithromycin, tocilizumab, umifenovir, galidesivir atorvastatin, and Pulmozyme.

14. The method of claim 10 wherein said additional therapeutic agent is an antiviral selected from Abacavir, Aciclovir, Acyclovir, Adefovir, Alisporivir, Amantadine, Amprenavir, Arbidol, Asunaprevir, Atazanavir, Baloxavir marboxil, Beclabuvir, Boceprevir, Brincidofovir, Brivudine, Cabotegravir, Cidofovir, Cobicistat, Combivir, Daclatasvir, Darunavir, Dasabuvir, Delavirdine, Didanosine, Docosanol, Dolutegravir, Doravirine, Edoxudine, Efavirenz, Elvitegravir, Emtricitabine, Enfuvirtide, Entecavir, Etravirine, Famciclovir, Favipiravir, Filociclovir, Fomivirsen, Fosamprenavir, Foscamet, Galidesivir, Ganciclovir, Glecaprevir, Grazoprevir, lbacitabine, lbalizumab, Idoxuridine, Imiquimod, Imunovir, Indinavir, Interferon alfacon 1, Lamivudine, Laninamivir, octanoate, Letermovir, Lobucavir, Lopinavir, Loviride, Maraviroc, Maribavir, Methisazone, Moroxydine, Nelfinavir, Nevirapine, Nexavir, Nitazoxanide, N-methanocarbathymidine, Norvir, Oseltamivir, Oseltamivir phosphate, Paliviumab, Paritaprevir, Pegylated interferon, Pegylated interferon alfa 2a, Pegylated interferon alfa 2b, Penciclovir, Peramivir, Pibrentasvir, Pimodivir, Pleconaril, Podophyllotoxin, Raltegravir, Recombinant human interleukin-7, Remdesivir, Ribavirin, Rilpivirine, Rimantadine, Rintatolimod, Ritonavir, RSV IGIV, Saquinavir, Simeprevir, Sinecatechins, Sofosbuvir, Stavudine, Taribavirin, Tecovirimat, Telaprevir, Telbivudine, Tenofovir, Tenofovir alafenamide, Tenofovir disoproxil fumarate, Thymalfasin, Tipranavir, Trifluridine, Tromantadine, Umifenovir, Valaciclovir, Valacyclovir, Valganciclovir, Vaniprevir, VariZIG, Vicriviroc, Vidarabine, VZIG, Zalcitabine, Zanamivir, Zidovudine and Zinc gluconate.

15. The method of claim 10 wherein said additional therapeutic agent is selected from Actemra (tocilizumab), sotrovimab, baricitinib, and anti-SARS-CoV-2 monoclonal antibodies such as bamlanivimab, etesevimab, casirivimab, imdevimab, REGN-COV2 ((casirivimab with imdevimab), and regdanvimab and cortisteroids such as prednisone, methylprednisolone,or hydrocortisone.

16. The method of claim 1 wherein the cannabinoid compound, or derivative thereof is administered prior to occurrence of any disease symptoms.

17. The method of claim 1 wherein the cannabinoid compound, or derivative thereof is administered after infection with virus.

18. A method of relieving a symptom of an RNA virus infection in a subject, comprising administering to the subject a cannabinoid compound, or derivative thereof; provided the RNA virus is not hepatitis or influenza.

19. A method of treating an RNA virus-related disease in a subject, comprising administering to the subject a cannabinoid compound, or derivative thereof; provided the RNA virus is not hepatitis or influenza.

20. The method of claim 17 wherein the disease is COVID-19.

21. A method of providing cytoprotection in a patient in need of such treatment, comprising administering to the patient a pharmacologically effective amount of a cannabinoid.

22. Use of a cannabinoid compound, or derivative thereof; in the manufacture of a medicament for treating an RNA virus, provided the RNA virus is not hepatitis or influenza.
